(19) United States
(12) Patent Application Publication
(10) Pub. No.: US 2024/0400626 A1
(43) Pub. Date: Dec. 5, 2024

(54) COMPOSITIONS AND METHODS FOR THE TREATMENT OF P53-MEDIATED CANCERS

(71) Applicant: SOLA BIOSCIENCES LLC, Natick, MA (US)

(72) Inventors: Akinori Hishiya, Belmont, MA (US); Keizo Koya, Chestnut Hill, MA (US)

(21) Appl. No.: 18/698,820

(22) PCT Filed: Oct. 7, 2022
(86) PCT No.: PCT/US2022/077779

§ 371 Date: Apr. 5, 2024

Related U.S. Application Data
(60) Provisional application No. 63/253,759, filed on Oct. 8, 2021.

(51) Int. Cl. C07K 14/47; A61K 38/00; C07K 16/32; C12N 15/86

(52) U.S. Cl. CPC C07K 14/47; C07K 16/32; C12N 15/86; A61K 38/00; C07K 2317/622; C07K 2317/92; C07K 2319/02; C07K 2319/30; C12N 2750/14143

(57) ABSTRACT

A novel class of fusion proteins to recruit a cell's innate chaperone mechanism, specifically the Hsp70-mediated system, to specifically restore p53-function is disclosed.

### RELATED APPLICATION

This application claims priority to U.S. Provisional Application No. 63/253,759, filed Oct. 8, 2021, the entirety of which is incorporated herein by reference.

### SEQUENCE LISTING

The instant application contains a Sequence Listing which is submitted herewith in electronically readable XML format via EFS-Web, and is hereby incorporated by reference in its entirety. The electronic Sequence Listing file, filed electronically on Oct. 7, 2022, and named “269548-517365_Sequence-isting.XML”, has a file size of 142,953 bytes.

### FIELD

The present disclosure relates to the use of chaperone fusion proteins for the treatment of P53-mediated cancers.

### BACKGROUND

Cancer is one of the leading causes of death world-wide. Approximately 38.4% of men and women will be diagnosed with cancer at some point during their lifetimes (based on 2013-2015 data: NIH National Cancer Research). Decreasing the suffering of cancer will have an immense impact on the whole society for improvement of total quality of life.

Traditional cancer treatments include surgery, radiation, medications and other therapies. These approaches may cure a cancer, shrink a cancer or stop the progression of a cancer. However, once cancer invasion or metastasis is observed, systemic treatment with medications would become the first line of defense to ameliorate the symptoms and improve the quality of life. Although many drugs have been developed during the last few decades to treat cancers, not too many of them have been satisfactory. Recent development using immuno-therapy is very exciting, but much work has yet to be done. The main reason for the difficulty associated with cancer treatment is that cancer (tumor) cells are derived from normal cells and they share many characteristics which make it difficult for drugs to specifically recognize only tumor cells. Yet decades' efforts by countless scientists have identified many unique features in common in cancer cells. For instance, it has been known that more than 50% of human cancers have mutations in the p53 gene. p53 exerts its main function as a transcription factor that induces cell cycle arrest, senescence, and apoptosis in response to genotoxic stress signals (Vogelstein, B. et al., (2000) Nature 408, 307-310; Petitjean, A. et al., (2007) Oncogene 26, 2157-2165). Owing to a high frequency of p53 mutations in cancers, one assumes that a promising strategy in cancer therapy can be developed by intervening in p53 mutants. But that promise has yet to be realized.

All proteins expressed within a cell need to correctly fold into their intended structures in order to function properly. A growing number of diseases and disorders are shown to be associated with inappropriate folding of proteins and/or inappropriate deposition and aggregation of proteins and lipoproteins as well as infectious proteinaceous substances. Also known as a conformational disease or proteopathy, examples of diseases caused by misfolding include Alzheimer's disease (AD), amyotrophic lateral sclerosis (ALS), and frontotemporal lobar dementia (FTLD). The mutant protein aggregates in cells causing typical cytotoxic cellular inclusion bodies.

Wild-type p53 protein contains 393 amino acids and is composed of several structural and functional domains. The N-terminal region consists of transactivation domain and a proline-rich region. Tetramerization domain and basic domain are located at the C terminus (Romer, L. et al., (2006) Angew Chem Int Ed Engl 45, 6440-6460; Joerger, A. C., and Fersht, A. R. (2008) Annu Rev Biochem 77, 557-582; Joerger, A. C., and Fersht, A. R. (2010) Cold Spring Harb Perspect Biol 2, a000919). It has been shown that a majority (˜74%) of p53 somatic mutations in cancer cells are missense-type mutations in the DNA binding domain called “hot spots”, leading to a single amino acid substitution in p53 protein, such as R175, G245, R248, R249, R273 and R282. These mutations can be divided into two classes: 1) DNA-contact mutations that result in loss of DNA-binding residues (R248, R273 and R282) and 2) conformational mutations that cause structural changes in p53 leading to misfolding of p53 proteins (R175, R245, and R249) (Strano, S. et al., (2007) Oncogene 26, 2212-2219; Freed-Pastor, W. A., and Prives, C. (2012) Genes Dev 26, 1268-1286). Since p53 normally functions as a tetramer, mutant p53 may abrogate the tumor suppression functions of remaining wild-type p53 counterpart or other p53 family members, in particular p63 and p73 through a ‘dominant-negative’ mechanism (Brosh, R., and Rotter, V. (2009) Nat Rev Cancer 9, 701-713; Muller, P. A., and Vousden, K. H. (2014) Cancer Cell 25, 304-317). Therefore, it has been thought that restoration of p53 function or suppression of mutant p53 activity has great potential as a novel effective strategy. It is until recently that compounds targeting the mutant oncogenic p53 proteins have emerged (Yu, X., Narayanan, S. et al., (2014) Apoptosis 19, 1055-1068). Aiming to screen compounds specifically targeting mutant p53 for growth suppression caused with R273H mutation, Bykov et al discovered that compound 2,2-bis(hydroxymethyl)-1-azabicyclo[2,2,2]octan-3-one, named PRIMA-1 (p53-reactivation and induction of massive apoptosis-1, APR-017), promoted refolding of p53 by modifying thiol groups in mutant p53 and induced several p53 target genes (Bykov, V. J. et al., (2002) Nat Med 8, 282-288; Bykov, V. J. et al., (2002) Carcinogenesis 23, 2011-2018). PRIMA-1 boosts apoptosis in human cancer cells with mutant p53 (Bykov, V. J. et al., (2005) Oncogene 24, 3484-3491; Lambert, J. M. et al., (2009) Cancer Cell 15, 376-388), becoming one of the first compounds that have been advanced to clinical trial phase I and phase II for hematologic malignancies and prostate cancer.

Cancer-related missense mutations in the p53 gene often increase the free energy of the protein, leading to its conformational change that divides mutant type of p53 from wild-type p53. Suppression of such mutant p53 is one of the promising strategies to regain p53 function. In line with this strategy, it has been shown that the refolding of mutant p53 by lowering temperature or chemical chaperones such as glycerol restore normal p53 function (Brown, C. R. et al., (1997) J Clin Invest 99, 1432-1444; Ohnishi, T. et al., (1999) Int J Radiat Biol 75, 1095-1098; Ohnishi, T., Ohnishi, K. et al., (1999) Radiat Res 151, 498-500).

The heat shock 70 kDa proteins (referred to herein as “Hsp70s”) constitute a ubiquitous class of chaperone proteins in the cells of a wide variety of species (Tavaria et al., (1996) Cell Stress Chaperones 1, 23-28). Hsp70 requires assistant proteins called co-chaperone proteins, such as J domain proteins and nucleotide exchange factors (NEFs) (Hartl et al., (2009) Nat Struct Mol Biol 16, 574-581), in order to function. In the current model of Hsp70 chaperone machinery for folding proteins, Hsp70 cycles between ATP- and ADP-bound states, and a J domain protein binds to another protein in need of folding or refolding (referred to as a “client protein”), interacting with the ATP-bound form of Hsp70 (Hsp70-ATP) (Young (2010) Biochem Cell Biol 88, 291-300; Mayer, (2010) Mol Cell 39, 321-331). Binding of the J domain protein-client complex to Hsp70-ATP stimulates ATP hydrolysis, which causes a conformational change in the Hsp70 protein, closing a helical lid and, thereby, stabilizing the interaction between the client protein with Hsp70-ADP, as well as eliciting the release of the J domain protein that is then free to bind to another client protein.

Therefore, according to this model, J domain proteins play a critical role within the Hsp70 machinery by acting as a bridge, and facilitating the capture and submission of a wide variety of client proteins into the Hsp70 machinery to promote folding or refolding into the proper conformation (Kampinga & Craig (2010) Nat Rev Mol Cell Biol 11, 579-592). The J domain family is widely conserved in species ranging from prokaryotes (DnaJ protein) to eukaryotes (Hsp40 protein family). The J domain (about 60-80 aa) is composed of four helices: I, II, III, and IV. Helices II and III are connected via a flexible loop containing an “HPD motif”, which is highly conserved across J domains and thought to be critical for activity (Tsai & Douglas, (1996) J Biol Chem 271, 9347-9354). Mutations within the HPD sequence has been found to abolish J domain function.

Given the context provided above for proteopathies, it seems clear that reducing the level of misfolded proteins could serve as a means to treat, prevent or otherwise ameliorate the symptoms of these devastating disorders and that, recruitment of a cell's innate ability to repair protein misfolding would be a logical choice to pursue.

### SUMMARY OF THE INVENTION

The inventors have developed a novel class of fusion proteins to recruit a cell's innate chaperone mechanism, specifically the Hsp70-mediated system, to specifically reduce levels of, or restore function of mutant p53. Unlike in previous studies by the inventors using fusion proteins comprising fragments of a Hsp40 protein (also called J proteins), a co-chaperone that interacts with Hsp70, to enhance protein secretion and expression, the present study employs J domain-containing fusion proteins for the purpose of reducing the levels of, or restoring the function of mutant p53 proteins. In this context, the inventors have made the surprising discovery that the elements of J domain required for function is quite distinct from use of J domains in enhancing protein expression and secretion, demonstrating a distinct mechanism for the mode of action of the present fusion proteins. The fusion proteins described herein comprise a J domain and a domain that has affinity for p53. The presence of the p53-binding domain within the fusion protein results in specific restoration of mutant p53 proteins.
E1. Therefore, in a first aspect, disclosed herein is an isolated fusion protein comprising a J domain of a J protein and a p53-binding domain.
E2. The fusion protein of E1, wherein the J domain of a J protein is of eukaryotic origin.
E3. The fusion protein of any one of E1-E2, wherein the J domain of a J protein is of human origin.
E4. The fusion protein of any one of E1-E3, wherein the J domain of a J protein is cytosolically localized.
E5. The fusion protein of any one of E1-E4, wherein the J domain of a J protein is selected from the group consisting of SEQ ID Nos: 1-50.
E6. The fusion protein of any one of E1-E5, wherein the J domain comprises the sequence selected from the group consisting of SEQ ID NOs: 1, 5, 6, 10, 16, 24, 25, 31 and 49.
E7. The fusion protein of any one of E1-E6, wherein the J domain comprises the sequence of SEQ ID NO: 5.
E8. The fusion protein of any one of E1-E6, wherein the J domain comprises the sequence of SEQ ID NO:10.
E9. The fusion protein of any one of E1-E6, wherein the J domain comprises the sequence of SEQ ID NO: 16.
E10. The fusion protein of any one of E1-E6, wherein the J domain comprises the sequence of SEQ ID NO: 25.
E11. The fusion protein of any one of E1-E6, wherein the J domain comprises the sequence of SEQ ID NO: 31.
E12. The fusion protein of any one of E1-E11, wherein the p53-binding domain has a K_{D} for p53 of 1 μM or less, for example, 300 nM or less, 100 nM or less, 30 nM or less, 10 nM or less, for example when measured using an ELISA assay.
E13. The fusion protein of any one of E1-E12, wherein the p53-binding domain comprises the sequence selected from the group consisting of SEQ ID NOs: 51-56.
E14. The fusion protein of any one of E1-E13, wherein the p53-binding domain comprises the sequence of SEQ ID NO:51-53.
E15. The fusion protein of any one of E1-E13, wherein the p53-binding domain comprises the sequence of SEQ ID NO:51.
E16. The fusion protein of any one of E1-E13, wherein the p53-binding domain comprises the sequence of SEQ ID NO:52.
E17. The fusion protein of any one of E1-E16, comprising a plurality of p53-binding domains.
E18. The fusion protein of any one of E1-E17, consisting of two p53-binding domains.
E19. The fusion protein of any one of E1-E18, consisting of three p53-binding domains.
E20. The fusion protein of any one of E1-E19, comprising one of the following constructs:

| | |
|---|---|
| a. | DNAJ-X-T, |
| b. | DNAJ-X-T-X-T, |
| c. | DNAJ-X-T-X-T-X-T, |
| d. | T-X-DNAJ, |
| e. | T-X-T-X-DNAJ, |
| f. | T-X-T-X-T-X-DNAJ, |
| g. | T-X-DNAJ-X-T, |
| h. | T-X-DNAJ-X-T-X-T, |
| i. | TDNAJ-X-TTTTTDNAJ-X-T, |
| j. | T-X-T-X-DNAJ-X-TT, |
| k. | TTDNAJ-X-T-X-TTTTTDNAJ-X-T, |
| l. | T-X-T-X-DNAJ-X-T-X-T-X-T, |
| m. | T-X-T-X-T-X-DNAJ-X-T, |
| n. | T-X-T-X-T-X-DNAJ-X-T-X-T, |
| o. | T-X-T-X-T-X-DNAJ-X-T-X-T-X-T, |
| p. | DnaJ-X-DnaJ-X-T-X-T, |
| q. | T-X-DnaJ-X-DnaJ, |
| r. | T-X-T-X-DnaJ-X-DnaJ, |
| and | |
| s. | T-X-TDnaJ-X-TDnaJ-X-TTTT |

wherein,
T is a p53-binding domain,
DNAJ is a J domain of a J protein, and
X is an optional linker.
E21. The fusion protein of any one of E1-E20, wherein the fusion protein comprises the J domain sequence of SEQ ID NO: 5 and the p53-binding domain sequence of SEQ ID NO: 52.
E22. The fusion protein of any one of E1-E21, wherein the fusion protein comprises the J domain sequence of SEQ ID NO: 5 and two copies of the p53-binding domain sequence of SEQ ID NO: 52.
E23. The fusion protein of any one of E1-E22, wherein the fusion protein comprises the sequence selected from the group consisting of SEQ ID NOs: 80-91, 100-107.
E24. The fusion protein of any one of E1-E23, wherein the fusion protein comprises the sequence selected from the group consisting of SEQ ID NOs: 80.
E25. The fusion protein of any one of E1-E23, wherein the fusion protein comprises the sequence of SEQ ID NO: 81.
E26. The fusion protein of any one of E1-E23, wherein the fusion protein comprises the sequence of SEQ ID NO: 82.
E27. The fusion protein of any one of E1-E23, wherein the fusion protein comprises the sequence of SEQ ID NO: 83.
E28. The fusion protein of any one of E1-E23, wherein the fusion protein comprises the sequence of SEQ ID NO: 84.
E29. The fusion protein of any one of E1-E23, wherein the fusion protein comprises the sequence of SEQ ID NO: 85.
E30. The fusion protein of any one of E1-E23, wherein the fusion protein comprises the sequence of SEQ ID NO: 86.
E31. The fusion protein of any one of E1-E23, wherein the fusion protein comprises the sequence of SEQ ID NO: 87.
E32. The fusion protein of any one of E1-E23, wherein the fusion protein comprises the sequence of SEQ ID NO: 88.
E33. The fusion protein of any one of E1-E23, wherein the fusion protein comprises the sequence of SEQ ID NO: 89.
E34. The fusion protein of any one of E1-E23, wherein the fusion protein comprises the sequence of SEQ ID NO: 90.
E35. The fusion protein of any one of E1-E23, wherein the fusion protein comprises the sequence of SEQ ID NO: 91.
E36. The fusion protein of any one of E1-E23, wherein the fusion protein comprises the sequence of SEQ ID NO: 100.
E37. The fusion protein of any one of E1-E23, wherein the fusion protein comprises the sequence of SEQ ID NO: 101.
E38. The fusion protein of any one of E1-E23, wherein the fusion protein comprises the sequence of SEQ ID NO: 102.
E39. The fusion protein of any one of E1-E23, wherein the fusion protein comprises the sequence of SEQ ID NO: 103.
E40. The fusion protein of any one of E1-E23, wherein the fusion protein comprises the sequence of SEQ ID NO: 104.
E41. The fusion protein of any one of E1-E23, wherein the fusion protein comprises the sequence of SEQ ID NO: 105.
E42. The fusion protein of any one of E1-E23, wherein the fusion protein comprises the sequence of SEQ ID NO: 106.
E43. The fusion protein of any one of E1-E23, wherein the fusion protein comprises the sequence of SEQ ID NO: 107.
E44. The fusion protein of any one of E1-E43, further comprising a targeting reagent.
E45. The fusion protein of any one of E1-E44, further comprising an epitope.
E46. The fusion protein of E45, wherein the epitope is a polypeptide selected from the group consisting of SEQ ID NOs:68-74.
E47. The fusion protein of any one of E1-E46, further comprising a cell-penetrating agent.
E48. The fusion protein of E47, wherein the cell-penetrating agent is selected from the group consisting of SEQ ID NOs: 75-78.
E49. The fusion protein of any one of E1-E48, further comprising a signal sequence.
E50. The fusion protein of E49, wherein the signal sequence comprises the peptide sequence selected from the group consisting of SEQ ID NOs: 94-96.
E51. The fusion protein of any one of E1-E50, which is capable of restoring p53 functions in a cell.
E52. The fusion protein of any one of E1-E51, which is capable of reducing p53-mediated neoplasm.
E53. A nucleic acid sequence encoding the fusion protein of any one of E1-E52.
E54. The nucleic acid sequence of E53, wherein said nucleic acid is DNA.
E55. The nucleic acid sequence of any one of E54, wherein said nucleic acid is RNA.
E56. The nucleic acid sequence of any one of E53-E55, wherein said nucleic acid comprises at least one modified nucleic acid.
E57. The nucleic acid sequence of any one of E53-E56, further comprising a promoter region, 5′ UTR, 3′ UTR such as poly(A) signal.
E58. The nucleic acid sequence of E57, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.
E59. A vector comprising the nucleic acid sequence of any one of E53-E58.
E60. The vector of E59, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.
E61. The vector of E59 or E60, wherein the vector is an AAV.
E62. A virus particle comprising a capsid and the vector of any one of E59-E61.
E63. The virus particle of E62, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.
E64. The virus particle of E62 or E63, wherein the capsid is selected from the group consisting of AAV2, AAV5, AAV8, AAV9 and AAVrh10.
E65. The virus particle of any one of E62-E64, wherein the capsid is AAV2.
E66. The virus particle of any one of E62-E64, wherein the capsid is AAV5.
E67. The virus particle of any one of E62-E64, wherein the capsid is AAV8.
E68. The virus particle of any one of E62-E64, wherein the capsid is AAV9.
E69. The virus particle of any one of E62-E64, wherein the capsid is AAV rh10.
E70. A pharmaceutical composition comprising an agent selected from the group consisting of the fusion protein of any one of E1-E52, a cell expressing the fusion protein of E1-E52, the nucleic acid of any one of E53-E58, the vector of any one of E59-E61, the virus particle of any one of E62-E69, and a pharmaceutically acceptable carrier or excipient.
E71. A method of reducing toxicity of a p53 protein in a cell, comprising contacting said cell with an effective amount of one or more agents selected from the group consisting of the fusion protein of any one of E1-E52, a cell expressing the fusion protein of E1-E52, the nucleic acid of any one of E53-E58, the vector of any one of E59-E61, the virus particle of any one of E62-E69, and the pharmaceutically composition of E70.
E72. The method of E71, wherein the cell is in a subject.
E73. The method of E72, wherein the subject is a human.
E74. The method of any one of E71-E73, wherein subject is identified as having cancer.
E75. The method of E74, wherein the cancer is selected from the group consisting malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.
E76. The method of any one of E71-E75, wherein there is a reduction in the amount of aberrant p53 protein in the cell when compared with a control cell.
E77. A method of treating, preventing, or delaying the progression of a cancer in a subject in need thereof, the method comprising administering an effective amount of one or more agents selected from the group consisting of the fusion protein of any one of E1-E52, a cell expressing the fusion protein of E1-E52, the nucleic acid of any one of E53-E58, the vector of any one of E59-E61, the virus particle of any one of E62-E69, and the pharmaceutically composition of E70.
E78. The method of E77, wherein the p53 disease is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.
E79. Use of one or more of the fusion protein of any one of E1-E52, a cell expressing the fusion protein of E1-E52, the nucleic acid of any one of E53-E58, the vector of any one of E59-E61, the virus particle of any one of E62-E69, and the pharmaceutically composition of E70, in the preparation of a medicament useful for the prevention or delay of progression of a p53 disease in a subject.
E80. The use of E79, wherein the p53 disease is cancer.
E81. The Use of E80, wherein the cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

### DESCRIPTION OF THE FIGURES

FIG. 1A shows a Clustal Omega sequence alignment of representative human J domain sequences. The highly conserved HPD domain is shown in the highlighted box.

FIG. 1B shows a Clustal Omega sequence alignment of representative human J domain sequences.

FIG. 2 shows some illustrative fusion protein constructs comprising a J domain and p53-binding domains.

FIG. 3 shows the effect of expressing wildtype or R175 or R249S mutants of p53, either alone or co-expressing Construct-2, on immunodetectable levels of p53 or p21, either in the absence of presence of 100 nM doxorubicin.

FIG. 4 shows the ELISA-based quantitation of p21 in HCT-116 cells stably transfected with the R249S mutant of p53, either alone or in the presence of Construct-2, treated with or without 1 μM doxorubicin.

FIG. 5 shows effects of expressing Construct-2 in HCT-116 cells stably transfected with constructs expressing either the R175H or R249S mutants of p53 on cytotoxicity, using LDH activity as a surrogate for cytotoxicity.

FIG. 6 shows the effects of expressing Construct 3 in cells transfected with constructs expressing either wildtype, R175H or R249 mutants of p53 on cytotoxicity, using LDH activity as an indicator of cytotoxicity.

FIG. 7 the effects of expressing Construct 16 or Construct 17 in cells transfected with constructs expressing either wildtype, R175H or R249 mutants of p53 on cytotoxicity, using LDH activity as an indicator of cytotoxicity.

### DEFINITIONS

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “p53 disorder” or “p53-mediated disease”, as herein defined refers to disorders associated with p53-mediated neoplasia, particularly caused by p53 mutant protein. Examples of p53 disorders include, but are not limited to preferably referring to cancers and other neoplasms, including but not limited to: malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “operably linked” refers to a juxtaposition of described components wherein the components are in a relationship permitting them to function in their intended manner. A control sequence “operably linked” to a coding sequence is ligated in such a way that expression of the coding sequence is achieved under conditions compatible with the control sequences. “Operably linked” sequences may include both expression control sequences that are contiguous with the gene of interest and expression control sequences that act in trans or at a distance to control the gene of interest. The term “expression control sequence” refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (such as, a Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing and can also include additional components whose presence is advantageous, for example, leader sequences and fusion partner sequences. Unless stated otherwise, a description or statement herein of inserting a nucleic acid molecule encoding a fusion protein of the invention into an expression vector means that the inserted nucleic acid has also been operably linked within the vector to a functional promoter and other transcriptional and translational control elements required for expression of the encoded fusion protein when the expression vector containing the inserted nucleic acid molecule is introduced into compatible host cells or compatible cells of an organism.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The terms “disease” and “disorder” are used interchangeably to indicate a pathological state identified according to acceptable medical standards and practices in the art.

As used herein, the term “effective amount” refers to the amount of a therapy that is sufficient to reduce or ameliorate the severity and/or duration of a disease or one or more symptoms thereof; to prevent the advancement of a detrimental or pathological state; to cause regression of a pathological state; to prevent recurrence, development, onset, or progression of one or more symptoms associated with a pathological state; to detect a disorder; or to enhance or improve the prophylactic or therapeutic effect(s) of a therapy (e.g., the administration of another prophylactic or therapeutic agent).

As used herein, the term “J domain” refers to a fragment which retains the ability to accelerate the intrinsic ATPase catalytic activity of Hsp70 and its cognate. The J domains of a variety of J proteins have been determined (see, for example, Kampinga et al. (2010) Nat. Rev., 11: 579-592; Hennessy et al. (2005) Protein Science, 14: 1697-1709, each of which is incorporated by reference in its entirety), and are characterized by a number of hallmarks: which is characterized by four α-helices (I, II, III, IV) and usually having the highly conserved tripeptide sequence motif of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is necessary for stimulation of Hsp70 ATPase activity. As used herein, the term “J domain” is meant to include natural J domain sequences and functional variants thereof which retain the ability to accelerate Hsp70 intrinsic ATPase activity, which can be measured using methods well known in the art (see, for example, Horne et al. (2010) J. Biol. Chem., 285, 21679-21688, which is incorporated herein by reference in its entirety). A non-limiting list of human J domains is provided in Table 1.

### DETAILED DESCRIPTION

The present inventors have found that certain contacting cells with a fusion protein construct comprising a J domain of a J protein and a p53-binding domain have the unexpected effect of restoring mutant p53 functions. Mutant p53 are believed to cause a number of devastating neoplasia. Accordingly, useful compositions and methods to treat p53 disorders, e.g., in a subject in need thereof, are provided herein.

To overcome issues associated with chaperone-based therapies, we investigated whether it would be possible to design artificial chaperone proteins with high specificity. We designed a series of fusion protein constructs comprising an effector domain for Hsp70 binding/activation (J domain sequence), and a domain conferring specificity to p53 proteins. The resulting fusion proteins act to accelerate the intrinsic ATPase catalytic activity of Hsp70 and its cognate, resulting in increased protein folding, restored function and/or accelerated clearance.

### I. Fusion Protein Constructs

a. J Domains Useful in the Invention

J domains of a variety of J proteins have been determined. See, for example, Kampinga et al., Nat. Rev., 11: 579-592 (2010); Hennessy et al., Protein Science, 14:1697-1709 (2005). A J domain useful in preparing a fusion protein of the invention has the key defining features of a J domain which principally accelerates HSP70 ATPase activity. Accordingly, an isolated J domain useful in the invention comprises a polypeptide domain, which is characterized by four α-helices (I, II, III, IV) and usually having the highly conserved tripeptide sequence of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is fundamental to primitive activity. Representative J domains include, but are not limited, a J domain of a DnaJB1, DnaJB2, DnaJB6, DnaJC6, a J domain of a large T antigen of SV40, and a J domain of a mammalian cysteine string protein (CSP-α). The amino acid sequences for these and other J domains that may be used in fusion proteins of the invention are provided in Table 1. The conserved HPD motif is highlighted in bold. In one embodiment, the fusion protein disclosed herein comprises a J domain selected from the group consisting of SEQ ID NOs: 1-50. In another embodiment, the fusion protein disclosed herein comprises a J domain comprising the consensus HPD motif. In one particular embodiment, selected from the group consisting of SEQ ID NOs: 1-15, 17-50. In a particular embodiment, the fusion protein comprises a J domain selected from the group consisting of SEQ ID NOs: 1, 5, 6, 10, 16, 24, 25, 31 and 49.

TABLE 1
Representative Human J Domain Sequences

| Protein Name | SEQ ID NO: | Length | J domain amino acid sequence |
|---|---|---|---|
| DNAJA1 | 1 | 63 | TYYDVLGVKPNATQEELKKAYRKLALKYHPDKNPNEGEKFK |
| | | | QISQAYEVLSDAKKRELYDKGG |
| DNAJA2 | 2 | 63 | KLYDILGVPPGASENELKKAYRKLAKEYHPDKNPNAGDKEK |
| | | | EISFAYEVLSNPEKRELYDRYG |
| DNAJA3 | 3 | 66 | DYYQILGVPRNASQKEIKKAYYQLAKKYHPDTNKDDPKAKE |
| | | | KFSQLAEAYEVLSDEVKRKQYDAYG |
| DNAJA4 | 4 | 67 | ETQYYDILGVKPSASPEEIKKAYRKLALKYHPDKNPDEGEK |
| | | | FKLISQAYEVLSDPKKRDVYDQGGEQ |
| DNAJB1 | 5 | 69 | GKDYYQTLGLARGASDEEIKRAYRRQALRYHPDKNKEPGAE |
| | | | EKFKEIAEAYDVLSDPRKREIFDRYGEE |
| DNAJB2 | 6 | 70 | ASYYEILDVPRSASADDIKKAYRRKALQWHPDKNPDNKEFA |
| | | | EKKFKEVAEAYEVLSDKHKREIYDRYGRE |
| DNAJB3 | 7 | 69 | MVDYYEVLDVPRQASSEAIKKAYRKLALKWHPDKNPENKEE |
| | | | AERRFKQVAEAYEVLSDAKKRDIYDRYG |
| DNAJB4 | 8 | 69 | GKDYYCILGIEKGASDEDIKKAYRKQALKFHPDKNKSPQAE |
| | | | EKFKEVAEAYEVLSDPKKREIYDQFGEE |
| DNAJB5 | 9 | 65 | DYYKILGIPSGANEDEIKKAYRKMALKYHPDKNKEPNAEEK |
| | | | FKEIAEAYDVLSDPKKRGLYDQYG |
| DNAJB6 | 10 | 68 | VDYYEVLGVQRHASPEDIKKAYRKLALKWHPDKNPENKEEA |
| | | | ERKEKQVAEAYEVLSDAKKRDIYDKYG |
| DNAJB7 | 11 | 67 | DYYEVLGLQRYASPEDIKKAYHKVALKWHPDKNPENKEEAE |
| | | | RKFKEVAEAYEVLSNDEKRDIYDKYG |
| DNAJB8 | 12 | 67 | NYYEVLGVQASASPEDIKKAYRKLALRWHPDKNPDNKEEAE |
| | | | KKFKLVSEAYEVLSDSKKRSLYDRAG |
| DNAJB9 | 13 | 65 | SYYDILGVPKSASERQIKKAFHKLAMKYHPDKNKSPDAEAK |
| | | | FREIAEAYETLSDANRRKEYDTLG |
| DNAJB11 | 14 | 66 | DFYKILGVPRSASIKDIKKAYRKLALQLHPDRNPDDPQAQE |
| | | | KFQDLGAAYEVLSDSEKRKQYDTYG |
| DNAJB12 | 15 | 65 | YEILGVSRGASDEDLKKAYRRLALKFHPDKNHAPGATEAFK |
| | | | AIGTAYAVLSNPEKRKQYDQFGDD |
| DNAJB13 | 16 | 66 | DYYSVLGITRNSEDAQIKQAYRRLALKHHPLKSNEPSSAEI |
| | | | FRQIAEAYDVLSDPMKRGIYDKFG |
| DNAJB14 | 17 | 65 | NYYEVLGVTKDAGDEDLKKAYRKLALKFHPDKNHAPGATDA |
| | | | FKKIGNAYAVLSNPEKRKQYDLTG |
| DNAJC1 | 18 | 65 | NFYQFLGVQQDASSADIRKAYRKLSLTLHPDKNKDENAETQ |
| | | | FRQLVAIYEVLKDDERRQRYDDIL |
| DNAJC2 | 19 | 74 | DHYAVLGLGHVRYKATQRQIKAAHKAMVLKHHPDKRKAAGE |
| | | | PIKEGDNDYFTCITKAYEMLSDPVKRRAFNSVD |
| DNAJC3 | 20 | 69 | DYYKILGVKRNAKKQEIIKAYRKLALQWHPDNFQNEEEKKK |
| | | | AEKKFIDIAAAKEVLSDPEMRKKEDDGE |
| DNAJC4 | 21 | 66 | TYYELLGVHPGASTEEVKRAFFSKSKELHPDRDPGNPSLHS |
| | | | RFVELSEAYRVLSREQSRRSYDDQL |
| DNAJC5 | 22 | 70 | GESLYHVLGLDKNATSDDIKKSYRKLALKYHPDKNPDNPEA |
| | | | ADKFKEINNAHAILTDATKRNIYDKYGSL |
| DNAJC5B | 23 | 66 | ALYEILGLHKGASNEEIKKTYRKLALKHHPDKNPDDPAATE |
| | | | KFKEINNAHAILTDISKRSIYDKYG |
| DNAJC6 | 24 | 65 | TKWKPVGMADLVTPEQVKKVYRKAVLVVHPDKATGQPYEQY |
| | | | AKMIFMELNDAWSEFENQGQKPLY |
| DNAJC7 | 25 | 71 | DYYKILGVDKNASEDEIKKAYRKRALMHHPDRHSGASAEVQ |
| | | | KEEEKKFKEVGEAFTILSDPKKKTRYDSGQ |
| DNAJC8 | 26 | 68 | NPFEVLQIDPEVTDEEIKKRFRQLSILVHPDKNQDDADRAQ |
| | | | KAFEAVDKAYKLLLDQEQKKRALDVIQ |
| DNAJC9 | 27 | 68 | DLYRVLGVRREASDGEVRRGYHKVSLQVHPDRVGEGDKEDA |
| | | | TRRFQILGKVYSVLSDREQRAVYDEQG |
| DNAJC10 | 28 | 66 | DFYSLLGVSKTASSREIRQAFKKLALKLHPDKNPNNPNAHG |
| | | | DELKINRAYEVLKDEDLRKKYDKYG |
| DNAJC11 | 29 | 69 | DYYSLLNVRREASSEELKAAYRRLCMLYHPDKHRDPELKSQ |
| | | | AERLENLVHQAYEVLSDPQTRAIYDIYG |
| DNAJC12 | 30 | 66 | DYYTLLGCDELSSVEQILAEFKVRALECHPDKHPENPKAVE |
| | | | TFQKLQKAKEILTNEESRARYDHWR |
| DNAJC13 | 31 | 66 | DAYEVLNLPQGQGPHDESKIRKAYFRLAQKYHPDKNPEGRD |
| | | | MFEKVNKAYEFLCTKSAKIVDGPDP |
| DNAJC14 | 32 | 65 | NPFHVLGVEATASDVELKKAYRQLAVMVHPDKNHHPRAEEA |
| | | | FKVLRAAWDIVSNAEKRKEYEMKR |
| DNAJC15 | 33 | 55 | EAGLILGVSPSAGKAKIRTAHRRVMILNHPDKGGSPYVAAK |
| | | | INEAKDLLETTTKH |
| DNAJC16 | 34 | 65 | DPYRVLGVSRTASQADIKKAYKKLAREWHPDKNKDPGAEDK |
| | | | FIQISKAYEILSNEEKRSNYDQYG |
| DNAJC17 | 35 | 66 | DLYALLGIEEKAADKEVKKAYRQKALSCHPDKNPDNPRAAE |
| | | | LFHQLSQALEVLTDAAARAAYDKVR |
| DNAJC18 | 36 | 65 | NYYEILGVSRDASDEELKKAYRKLALKFHPDKNCAPGATDA |
| | | | FKAIGNAFAVLSNPDKRLRYDEYG |
| DNAJC19 | 37 | 55 | EAALILGVSPTANKGKIRDAHRRIMLLNHPDKGGSPYIAAK |
| | | | INEAKDLLEGQAKK |
| DNAJC20 | 38 | 72 | DYFSLMDCNRSFRVDTAKLQHRYQQLQRLVHPDFFSQRSQT |
| | | | EKDFSEKHSTLVNDAYKTLLAPLSRGLYLLK |
| DNAJC21 | 39 | 67 | CHYEALGVRRDASEEELKKAYRKLALKWHPDKNLDNAAEAA |
| | | | EQFKLIQAAYDVLSDPQERAWYDNHR |
| DNAJC22 | 40 | 65 | LAYQVLGLSEGATNEEIHRSYQELVKVWHPDHNLDQTEEAQ |
| | | | RHFLEIQAAYEVLSQPRKPWGSRR |
| DNAJC23 | 41 | 62 | NPYEVLNLDPGATVAEIKKQYRLLSLKYHPDKGGDEVMEMR |
| | | | IAKAYAALTDEESRKNWEEFG |
| DNAJC24 | 42 | 72 | DWYSILGADPSANISDLKQKYQKLILMYHPDKQSTDVPAGT |
| | | | VEECVQKFIEIDQAWKILGNEETKREYDLQR |
| DNAJC25 | 43 | 76 | DCYEVLGVSRSAGKAEIARAYRQLARRYHPDRYRPQPGDEG |
| | | | PGRTPQSAEEAFLLVATAYETLKDEETRKDYDYML |
| DNAJC26 | 44 | 65 | SRWTPVGMADLVAPEQVKKHYRRAVLAVHPDKAAGQPYEQH |
| | | | AKMIFMELNDAWSEFENQGSRPLF |
| DNAJC27 | 45 | 57 | DSWDMLGVKPGASRDEVNKAYRKLAVLLHPDKCVAPGSEDA |
| | | | FKAVVNARTALLKNIK |
| DNAJC28 | 46 | 65 | EYYRLLNVEEGCSADEVRESFHKLAKQYHPDSGSNTADSAT |
| | | | FIRIEKAYRKVLSHVIEQTNASQS |
| DNAJC29 | 47 | 88 | ILKEVTSVVEQAWKLPESERKKIIRRLYLKWHPDKNPENHD |
| | | | IANEVFKHLQNEINRLEKQAFLDQNADRASRRTESTSASRE |
| | | | QSDKYS |
| DNAJC30 | 48 | 66 | ALYDLLGVPSTATQAQIKAAYYRQCFLYHPDRNSGSAEAAE |
| | | | RFTRISQAYVVLGSATLRRKYDRGL |
| SV40 | 49 | 64 | QLMDLLGLERSAWGNIPLMRKAYLKKCKEFHPDKGGDEEKM |
| Jdomain | | | KKMNTLYKKMEDGVKYAHQPDFG |
| Bacterial | 50 | 70 | KQDYYEILGVSKTAEEREIRKAYKRLAMKYHPDRNQGDKEA |
| J-domain | | | EAKFKEIKEAYEVLTDSQKRAAYDQYGHA |

b. p53-Binding Domain

The fusion protein also comprises at least one p53-binding domain. The p53-binding domain can be a single chain polypeptide, or a multimeric polypeptide joined with the J domain to form the fusion protein.

It is ideal that the p53-binding domain possesses a sufficient affinity to be able to bind the p53 protein when present at a pathological level within cells. Therefore, in one embodiment, the fusion protein comprises a p53-binding domain that has a K_{D} for p53 reporter construct of, for example, 2 μM or less, 1 μM or less, 500 nM or less, 300 nM or less, 100 nM or less, 30 nM or less when tested by ELISA on 96 well microtiter plates.

It is noted that the HSP70 machinery is believed to only engage misfolded proteins. However, in some cases, it may be preferable for the fusion proteins to engage only the misfolded forms of p53. Therefore, in some embodiments, the p53-binding domain binds preferentially to the misfolded forms of p53. Although not necessary, the ability of the p53-binding domain to have higher affinity for the misfolded form (for example, the R175, R245, and R249 mutant forms of p53) would allow the fusion proteins to more selectively engage only the pathogenic forms to the HSP70 machinery.

In another embodiment, the fusion protein also contemplates the use of the p53-binding domain that is chemically conjugated to the J domain. The p53-binding domain can be conjugated directly to the J domain. Alternatively, it can be conjugated to the J domain by a linker. For example, there are a large number of chemical cross-linking agents that are known to those skilled in the art and useful for cross-linking the p53-binding domain to the J domain, or a targeting domain to a fusion protein comprising the p53-binding domain and J domain. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl(4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate·2 HCl (Forbes-Cori Disease) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al., (1990) Bioconj. Chem. 1:2-12, incorporated by reference herein.

TABLE 2
Examples of p53-binding domains

| Name | SEQ ID NO: | Length | Sequence |
|---|---|---|---|
| p53BP | 51 | 7 | NPNSAQG |
| CDB3 | 52 | 9 | REDEDEIEW |
| scFv1 | 53 | 256 | QVKLQQSGAELAKPGASVKMSCKTSGYTFTSYWMNWVKQ |
| (ME1) | | | RPGQGLEWIGYINPTTGYTKYNQKFKDKATLTADKSSST |
| | | | AYMQLSSLTNVDSAVYYCTTGYSYFDYWGQGTTVTVSSG |
| | | | GGGSGGGGSGGGGSDIELTQSPAIMSASPGEKVTITCSA |
| | | | SSSVNYMHWFQQKPGTSPKLWISSTSNLASGVPARESGS |
| | | | GSGTSYSLTISRMEAEDAATYYCQQRSSYPYTFGGGTKL |
| | | | QIKRAAAGAPVPYPDPLEPRAA |
| scFv2 | 54 | 249 | QVKLQESGAELVRSGASVNLSCTASGFNIKDYYMHWVKQ |
| (11D3) | | | RPEEGLEWIGYIDPESGETEYAPNFQGKATVTADTSSNT |
| | | | AYLHLSSLTSEDTTVYYCNAVIYYEYDGYALDYWGQGTT |
| | | | VTVSSGGGGSGGGGSGGGGSDIELTQSPSSLAVSAGEKV |
| | | | AMSCKSSQSLFNSRTRKNYLAWYQQKPGQSPKVLIYWAS |
| | | | TRESGVPDRFTGSGSGTDFTLTISSVQAEDLAVYYCKQS |
| | | | YNLPTFGGGTKLEIK |
| scFv3 | 55 | 243 | QVQLQQSGAELVRSGASVKLSCTASGFNIKDYYMHWVKQ |
| (scFv421) | | | RPEQGLEWIGWIDPENGDTEYAPKFQGKATMTADTSSNT |
| | | | AYLQLSSLASEDTAVYYCNFYGDALDYWGQGTTVTVSSG |
| | | | GGGSGGGGSGGGGSDVLMTQTPLTLSVTIGQPASISCKS |
| | | | SQSLLDSDGKTYLNWLLQRPGQSPKRLIYLVSKLDSGVP |
| | | | DRFTGSGSGTDFTLKINRVEAEDLGVYYCWQGTHSPLTF |
| | | | GAGTKLEIK |
| scFv4 | 56 | 233 | QESGAELVRSGASVKLSCTTSGFNINDYYMHWVKKRPEQ |
| (Pab421) | | | GLEWIGRIDPENGDADMTRSSGVKATMTADTSSNTAYLQ |
| | | | LSSLTSEDTAVYYCNAGMDYWGQGTTVTVSSGGGGSGGR |
| | | | ASGGGGSDIELTQSPASLAVSLGQRATISCRASKSVSTS |
| | | | GYSYMHWNQQKPGQPPRLLIYLVSNLESGVPARFSGSGS |
| | | | GTDFTLNIHPVEEEDAATYYCQHIRELTRSEGGTKLEI |

c. Optional Linker

The fusion proteins described herein can optionally contain one or more linkers. Linkers can be peptidic or non-peptidic. The purpose of the linker is to provide, among other things, an adequate distance between functional domains within the protein (e.g., between the J domain and p53-binding domain, between tandem arrangements of p53-binding domains, between either the J domain and p53-binding domain and an optional targeting reagent, or between either the J domain and p53-binding domain and an optional detection domain or epitope) for optimal function of each of the domains. Clearly, a linker preferably does not interfere with the respective functions of the J domain, the target protein binding domain of a fusion protein according to the invention. A linker, if present in a fusion protein of the invention, can be selected to attenuate the cytotoxicity caused by target proteins (p53 proteins), and it may be omitted if direct attachment achieves a desired effect. Linkers present in a fusion protein of the invention may comprise one or more amino acids encoded by a nucleotide sequence present on a segment of nucleic acid in or around a cloning site of an expression vector into which is inserted in frame a nucleic acid segment encoding a protein domain or an entire fusion protein as described herein. In one embodiment, the peptide linker is between 1 amino acid and 20 amino acids in length. In another embodiment, the peptide linker is between 2 amino acids and 15 amino acids in length. In still another embodiment, the peptide linker is between 2 amino acids and 10 amino acids in length.

Selecting one or more polypeptide linkers to produce a fusion protein according to the invention is within the knowledge and skill of practitioners in the art. See, for example, Arai et al., Protein Eng., 14(8): 529-532 (2001); Crasto et al., Protein Eng., 13(5): 309-314 (2000); George et al., Protein Eng., 15(11): 871-879 (2003); Robinson et al., Proc. Natl. Acad. Sci. USA, 95: 5929-5934 (1998), each of which is incorporated herein by reference in its entirety. Examples of linkers of two or more amino acids that may be used in preparing a fusion protein according to the invention, include, by are not limited to, those provided below in Table 3.

TABLE 3
Linker Sequences

| SEQ ID NO: | Length | Sequence |
|---|---|---|
| 57 | 2 | SR |
| 58 | 4 | GTGS |
| 59 | 5 | GLESR |
| 60 | 4 | GGSG |
| 61 | 5 | GGGGS |
| 62 | 5 | DIAAA |
| 63 | 9 | DIAAALESR |
| 64 | 15 | GGGGSGGGGSGGGGS |
| 65 | 11 | AEAAAKEAAAK |
| 66 | 15 | SGGGSGGGGSGGGGS |
| 67 | 25 | DIGGGGSGGGGSGGGGSGGGGSAAA |

d. Targeting Reagents

The fusion proteins disclosed herein can further comprise a targeting moiety. As used herein, the terms “targeting moiety” and “targeting reagent” are used interchangeably and refer to a substance associated with the fusion protein that enhances binding, transport, accumulation, residence time, bioavailability, or modifies biological activity or therapeutic effect of the fusion protein in a cell or in the body of a subject. A targeting moiety can have functionality at the tissue, cellular, and/or subcellular level. The targeting moiety can direct localization of the fusion protein to a particular cell, tissue or organ, or intracellular distribution, for example, upon administration of the fusion protein into a subject. In one embodiment, the targeting moiety is located at the N-terminus of the fusion protein. In another embodiment, the targeting moiety is located at the C-terminus of the fusion protein. In still another embodiment, the targeting moiety is located internally. In another embodiment, the targeting moiety is attached to the fusion protein via chemical conjugation.

The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxins, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a growth factor, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity of the modifies biological activity or therapeutic effect of the platform, or its associated ligand and/or active agent in the target cell or tissue, for example, neuronal cells, the central nervous system, and/or the peripheral nervous system. Thus, the targeting moiety can have specificity for cellular receptors associated with the central nervous system, or is otherwise associated with enhanced delivery to the CNS via the blood-brain barrier (BBB). Consequently, a ligand, as described above, can be both a ligand and a targeting moiety.

In some embodiments, the targeting moiety can be a cell-penetrating peptide, for example, as described in U.S. Pat. No. 10,111,965, which is incorporated by reference in its entirety. In another embodiment, the targeting moiety can be an antibody or an antigen-binding fragment or single-chain derivative thereof, for example, as described in U.S. Ser. No. 16/131,591, which is incorporated herein by reference in its entirety. In further embodiment, the targeting moiety can be a amino acid sequence for nuclear localization signal or nuclear export signal.

The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to core byway of covalent, non-covalent, or electrostatic interactions. In one embodiment, the targeting moiety is a peptide. In a particular embodiment, the targeting moiety is a peptide that is covalently attached to the N-terminus of the fusion protein.

e. Epitopes

In certain embodiments, the fusion protein of the present invention contains an optional epitope or tag, which can impart additional properties to the fusion protein. As used herein, the terms “epitope” and “tag” are used interchangeably to refer to an amino acid sequence, typically 300 amino acids or less in length, which is typically attached to the N-terminal or C-terminal end of the fusion protein. In one embodiment, the fusion protein of the present invention further comprises an epitope which is used to facilitate purification. Examples of such epitopes useful for purification, provided below in Table 4, include the human IgG1 Fc sequence (SEQ ID NO: 68), the FLAG epitope (DYKDDDDK, SEQ ID NO: 69), His6 epitope (SEQ ID NO: 70), c-myc (SEQ ID NO: 71), HA (SEQ ID NO: 72), V5 epitope (SEQ ID NO: 73), or glutathione-s-transferase (SEQ ID NO: 74). In another embodiment, the fusion protein of the present invention further comprises an epitope which is used to increase the half-life of the fusion protein when administered into a subject, for example a human. Examples of such epitopes useful for increasing half-life include the human Fc sequence. Therefore, in one particular embodiment, the fusion protein comprises, in addition to a J domain and p53-binding domain, a human Fc epitope. The epitope is positioned at the C-terminal end of the fusion protein.

TABLE 4
Representative Examples of Epitopes

| SEQ ID NO: | EPITOPE | LENGTH | SEQUENCE |
|---|---|---|---|
| 68 | Human lgG1 | 232 | EPKSCDKTHTCPPCPAPELLGGPSVFLFPPKPKDTLM |
| | Fc domain | | ISRTPEVTCVVVDVSHEDPEVKENWYVDGVEVHNAKT |
| | | | KPREEQYNSTYRVVSVLTVLHQDWLNGKEYKCKVSNK |
| | | | ALPAPIEKTISKAKGQPREPQVYTLPPSRDELTKNQV |
| | | | SLTCLVKGFYPSDIAVEWESNGQPENNYKTTPPVLDS |
| | | | DGSFFLYSKLTVDKSRWQQGNVFSCSVMHEALHNHYT |
| | | | QKSLSLSPGK |
| 69 | FLAG epitope | 8 | DYKDDDDK |
| 70 | His6 | 6 | HHHHHH |
| 71 | c-myc | 10 | EQKLISEEDL |
| 72 | HA | 9 | YPYDVPDYA |
| 73 | V5 epitope | 14 | GKPIPNPLLGLDST |
| 74 | Glutathione- | 220 | MSPILGYWKIKGLVQPTRLLLEYLEEKYEEHLYERDE |
| | S-transferase | | GDKWRNKKFELGLEFPNLPYYIDGDVKLTQSMAIIRY |
| | | | IADKHNMLGGCPKERAEISMLEGAVLDIRYGVSRIAY |
| | | | SKDFETLKVDFLSKLPEMLKMFEDRLCHKTYLNGDHV |
| | | | THPDFMLYDALDVVLYMDPMCLDAFPKLVCEKKRIEA |
| | | | IPQIDKYLKSSKYIAWPLQGWQATFGGGDHPPKSD |

f. Cell-Penetrating Peptides

In still other embodiments, the fusion protein described herein can further comprise a cell-penetrating peptide. Cell-penetrating peptides are known to carry a conjugated cargo, whether a small molecule, peptide, protein or nucleic acid, into cells. Non-limiting examples of cell-penetrating peptides in a fusion protein of the invention include, but are not limited to, a polycationic peptide, e.g., an HIV TAT peptide49-57, polyarginines, and penetratin pAntan(43-58), amphipathic peptide, e.g., pep-1, a hydrophobic peptide, e.g., a C405Y, and the like. See Table 5 below.

TABLE 5
Examples of Cell-Penetrating Peptides

| SEQ ID NO: | LENGTH | SEQUENCE |
|---|---|---|
| 75 | 9 | RKKRRQRRR |
| 76 | 15 | RQIKWFQNRRMKWKK |
| 77 | 21 | KETWWETWWTEWSQPKKKRKV |
| 78 | 17 | CSIPPEVKENKPFVYLI |

Therefore, in one embodiment, the fusion protein comprises a cell-penetrating peptide and a fusion protein, wherein the cell-penetrating peptide is selected from the group consisting of SEQ ID NOs: 75-78, and the fusion protein comprising a J domain and a p53 binding domain. In one embodiment, the fusion protein is selected from the group consisting of SEQ ID NOs: 80-91, 100-107. In another embodiment, the fusion protein comprises the signal sequence of SEQ ID NO: 75, and the fusion protein selected from the group consisting of SEQ ID NOs: 80-91, 100-107. In another embodiment, the fusion protein comprises the cell-penetrating peptide of SEQ ID NO: 76, and the fusion protein selected from the group consisting of SEQ ID NOs: 80-91, 100-107. In still another embodiment, the fusion protein comprises the cell-penetrating peptide of SEQ ID NO: 77, and the fusion protein selected from the group consisting of SEQ ID NOs: 80-91, 100-107. In yet another embodiment, the fusion protein comprises the cell-penetrating peptide of SEQ ID NO: 78, and the fusion protein selected from the group consisting of SEQ ID NOs: 80-91, 100-107. Cells expressing the fusion protein constructs with the cell-penetrating peptide can be administered to a subject, for example a human subject (e.g., a patient having or at risk of suffering from a p53 disorder). The fusion protein is secreted from the cells, which help restore p53 functions and/or aggregation.

g. Arrangement of J Domain and p53 Binding Domain

The fusion proteins described herein can be arranged in a multitude of ways. In one embodiment, the p53-binding domains attached to the C-terminal side of the J domain. In another embodiment, the p53-binding domains attached to the N-terminal side of the J domain. The p53-binding domain and the J domain, in either configuration, can optionally be separated via a linker as described above.

In some embodiments, the J domain can be attached to a plurality of p53-binding domains, for example, two p53-binding domains, three p53-binding domains, four p53-binding domains or more. The p53-binding domains can be attached to the N-terminal side of the J domain. Alternatively, the p53-binding domains can be attached to the C-terminal side of the J domain. In still another embodiment, the p53-binding domains can be attached on the N-terminal and C-terminal sides of the J domain. Each of the plurality of p53-binding domains can be the same p53-binding domain. In another embodiment, each of the plurality of p53-binding domains in the fusion protein can be different p53-binding domains (i.e., different sequences).

In some embodiments, the fusion proteins can comprise a structure selected from the following group:

| | |
|---|---|
| a. | DNAJ-X-T, |
| b. | DNAJ-X-T-X-T, |
| c. | DNAJ-X-T-X-T-X-T, |
| d. | T-X-DNAJ, |
| e. | T-X-T-X-DNAJ, |
| f. | T-X-T-X-T-X-DNAJ, |
| g. | T-X-DNAJ-X-T, |
| h. | T-X-DNAJ-X-T-X-T, |
| i. | T-X-T-X-DNAJ-X-TT |
| j. | TTDNAJ-X-T-X-TTTTTDNAJ-X-T |
| k. | T-X-T-X-DNAJ-X-T-X-T-X-T, |
| l. | T-X-T-X-T-X-DNAJ-X-T, |
| m. | T-X-T-X-T-X-DNAJ-X-T-X-T, |
| n. | T-X-T-X-T-X-DNAJ-X-T-X-T-X-T, |
| o. | DnaJ-X-DnaJ-X-T-X-T, |
| p. | T-X-DnaJ-X-DnaJ, |
| q. | T-X-T-X-DnaJ-X-DnaJ, |
| and | |
| r. | T-X-TDnaJ-X-TDnaJ-X-TTTT |

wherein,
T is a p53-binding domain,
DNAJ is a J domain of a J protein, and
X is an optional linker.

In one embodiment, the fusion protein comprises the J domain selected from the group consisting of SEQ ID NOs: 5, 6, 10, 24, and 31. In one particular embodiment, the fusion protein comprises the J domain of SEQ ID NO: 5.

In another embodiment, the p53-binding domain is selected from the group consisting of SEQ ID NOs:51-56. In one particular embodiment, the p53-binding domain is selected from the group consisting of SEQ ID NOs:51-52.

In still another embodiment, the fusion protein comprises the J domain of SEQ ID NO: 5, and the p53-binding domain of SEQ ID NO: 51. In another embodiment, the fusion protein comprises the J domain of SEQ ID NO: 5, and at least two copies of the p53-binding domain of SEQ ID NO: 52.

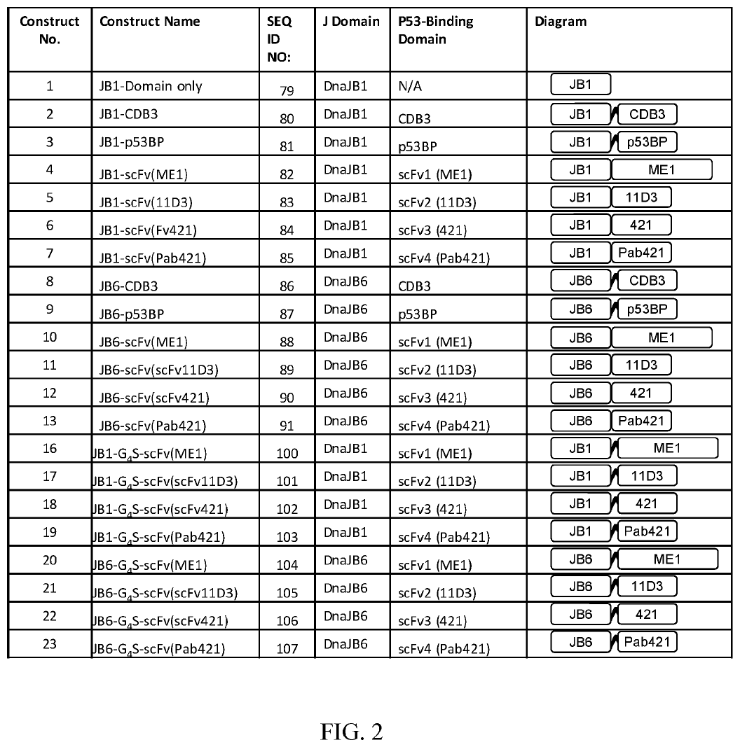

Non-limiting examples of fusion protein constructs comprising a J domain and p53-binding domain are depicted schematically in FIG. 2, and also shown below in Table 6. In another embodiment, the specific fusion protein construct is selected from the group consisting of SEQ ID NOs: 80-91.

TABLE 6
Fusion Protein Constructs and Control Constructs

| Construct | SEQ | Construct | | |
|---|---|---|---|---|
| No | ID NO: | Name | Length | Sequence |
| 1 | 79 | J-domain only | 75 | MGKDYYQTLGLARGASDEEIKRAYRRQALRYHPDKNKE |
| | | | | PGAEEKFKEIAEAYDVLSDPRKREIFDRYGEEGLKGS |
| 2 | 80 | JB1-CDB3 | 89 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSREDEDEIEW |
| 3 | 81 | JB1-p53BP | 87 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSNPNSAQG |
| 4 | 82 | JB1-scFv1 | 331 | MGKDYYQTLGLARGASDEEIKRAYRRQALRYHPDKNKE |
| | | | | PGAEEKFKEIAEAYDVLSDPRKREIFDRYGEEGLKGSQ |
| | | | | VKLQQSGAELAKPGASVKMSCKTSGYTFTSYWMNWVKQ |
| | | | | RPGQGLEWIGYINPTTGYTKYNQKFKDKATLTADKSSS |
| | | | | TAYMQLSSLTNVDSAVYYCTTGYSYFDYWGQGTTVTVS |
| | | | | SGGGGSGGGGSGGGGSDIELTQSPAIMSASPGEKVTIT |
| | | | | CSASSSVNYMHWFQQKPGTSPKLWISSTSNLASGVPAR |
| | | | | FSGSGSGTSYSLTISRMEAEDAATYYCQQRSSYPYTFG |
| | | | | GGTKLQIKRAAAGAPVPYPDPLEPRAA |
| 5 | 83 | JB1-scFv2 | 324 | MGKDYYQTLGLARGASDEEIKRAYRRQALRYHPDKNKE |
| | | | | PGAEEKFKEIAEAYDVLSDPRKREIFDRYGEEGLKGSQ |
| | | | | VKLQESGAELVRSGASVNLSCTASGFNIKDYYMHWVKQ |
| | | | | RPEEGLEWIGYIDPESGETEYAPNFQGKATVTADTSSN |
| | | | | TAYLHLSSLTSEDTTVYYCNAVIYYEYDGYALDYWGQG |
| | | | | TTVTVSSGGGGSGGGGSGGGGSDIELTQSPSSLAVSAG |
| | | | | EKVAMSCKSSQSLFNSRTRKNYLAWYQQKPGQSPKVLI |
| | | | | YWASTRESGVPDRFTGSGSGTDFTLTISSVQAEDLAVY |
| | | | | YCKQSYNLPTFGGGTKLEIK |
| 6 | 84 | JB1-scFv3 | 318 | MGKDYYQTLGLARGASDEEIKRAYRRQALRYHPDKNKE |
| | | | | PGAEEKFKEIAEAYDVLSDPRKREIFDRYGEEGLKGSQ |
| | | | | VQLQQSGAELVRSGASVKLSCTASGFNIKDYYMHWVKQ |
| | | | | RPEQGLEWIGWIDPENGDTEYAPKFQGKATMTADTSSN |
| | | | | TAYLQLSSLASEDTAVYYCNFYGDALDYWGQGTTVTVS |
| | | | | SGGGGSGGGGSGGGGSDVLMTQTPLTLSVTIGQPASIS |
| | | | | CKSSQSLLDSDGKTYLNWLLQRPGQSPKRLIYLVSKLD |
| | | | | SGVPDRFTGSGSGTDFTLKINRVEAEDLGVYYCWQGTH |
| | | | | SPLTFGAGTKLEIK |
| 7 | 85 | JB1-scFv4 | 308 | MGKDYYQTLGLARGASDEEIKRAYRRQALRYHPDKNKE |
| | | | | PGAEEKFKEIAEAYDVLSDPRKREIFDRYGEEGLKGSQ |
| | | | | ESGAELVRSGASVKLSCTTSGFNINDYYMHWVKKRPEQ |
| | | | | GLEWIGRIDPENGDADMTRSSGVKATMTADTSSNTAYL |
| | | | | QLSSLTSEDTAVYYCNAGMDYWGQGTTVTVSSGGGGSG |
| | | | | GRASGGGGSDIELTQSPASLAVSLGQRATISCRASKSV |
| | | | | STSGYSYMHWNQQKPGQPPRLLIYLVSNLESGVPARFS |
| | | | | GSGSGTDFTLNIHPVEEEDAATYYCQHIRELTRSEGGT |
| | | | | KLEI |
| 8 | 86 | JB6-CDB3 | 83 | MVDYYEVLGVQRHASPEDIKKAYRKLALKWHPDKNPEN |
| | | | | KEEAERKEKQVAEAYEVLSDAKKRDIYDKYGGGGGSRE |
| | | | | DEDEIEW |
| 9 | 87 | JB6-p53BP | 81 | MVDYYEVLGVQRHASPEDIKKAYRKLALKWHPDKNPEN |
| | | | | KEEAERKFKQVAEAYEVLSDAKKRDIYDKYGGGGGSNP |
| | | | | NSAQG |
| 10 | 88 | JB6-scFv1 | 325 | MVDYYEVLGVQRHASPEDIKKAYRKLALKWHPDKNPEN |
| | | | | KEEAERKEKQVAEAYEVLSDAKKRDIYDKYGQVKLQQS |
| | | | | GAELAKPGASVKMSCKTSGYTFTSYWMNWVKQRPGQGL |
| | | | | EWIGYINPTTGYTKYNQKFKDKATLTADKSSSTAYMQL |
| | | | | SSLTNVDSAVYYCTTGYSYFDYWGQGTTVTVSSGGGGS |
| | | | | GGGGGGGGSDIELTQSPAIMSASPGEKVTITCSASSS |
| | | | | VNYMHWFQQKPGTSPKLWISSTSNLASGVPARFSGSGS |
| | | | | GTSYSLTISRMEAEDAATYYCQQRSSYPYTFGGGTKLQ |
| | | | | IKRAAAGAPVPYPDPLEPRAA |
| 11 | 89 | JB6-scFv2 | 318 | MVDYYEVLGVQRHASPEDIKKAYRKLALKWHPDKNPEN |
| | | | | KEEAERKFKQVAEAYEVLSDAKKRDIYDKYGQVKLQES |
| | | | | GAELVRSGASVNLSCTASGFNIKDYYMHWVKQRPEEGL |
| | | | | EWIGYIDPESGETEYAPNFQGKATVTADTSSNTAYLHL |
| | | | | SSLTSEDTTVYYCNAVIYYEYDGYALDYWGQGTTVTVS |
| | | | | SGGGGSGGGGSGGGGSDIELTQSPSSLAVSAGEKVAMS |
| | | | | CKSSQSLFNSRTRKNYLAWYQQKPGQSPKVLIYWASTR |
| | | | | ESGVPDRFTGSGSGTDFTLTISSVQAEDLAVYYCKQSY |
| | | | | NLPTFGGGTKLEIK |
| 12 | 90 | JB6-scFv3 | 312 | MVDYYEVLGVQRHASPEDIKKAYRKLALKWHPDKNPEN |
| | | | | KEEAERKFKQVAEAYEVLSDAKKRDIYDKYGQVQLQQS |
| | | | | GAELVRSGASVKLSCTASGFNIKDYYMHWVKQRPEQGL |
| | | | | EWIGWIDPENGDTEYAPKFQGKATMTADTSSNTAYLQL |
| | | | | SSLASEDTAVYYCNFYGDALDYWGQGTTVTVSSGGGGS |
| | | | | GGGGSGGGGSDVLMTQTPLTLSVTIGQPASISCKSSQS |
| | | | | LLDSDGKTYLNWLLQRPGQSPKRLIYLVSKLDSGVPDR |
| | | | | FTGSGSGTDFTLKINRVEAEDLGVYYCWQGTHSPLTFG |
| | | | | AGTKLEIK |
| 13 | 91 | JB6-scFv4 | 302 | MVDYYEVLGVQRHASPEDIKKAYRKLALKWHPDKNPEN |
| | | | | KEEAERKFKQVAEAYEVLSDAKKRDIYDKYGQESGAEL |
| | | | | VRSGASVKLSCTTSGFNINDYYMHWVKKRPEQGLEWIG |
| | | | | RIDPENGDADMTRSSGVKATMTADTSSNTAYLQLSSLT |
| | | | | SEDTAVYYCNAGMDYWGQGTTVTVSSGGGGSGGRASGG |
| | | | | GGSDIELTQSPASLAVSLGQRATISCRASKSVSTSGYS |
| | | | | YMHWNQQKPGQPPRLLIYLVSNLESGVPARFSGSGSGT |
| | | | | DFTLNIHPVEEEDAATYYCQHIRELTRSEGGTKLEI |
| 16 | 100 | JB1-G4S-scFv1 | 336 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSQVKLQQSGAELAKPGASVKMSCKTSGYTFTSYWM |
| | | | | NWVKQRPGQGLEWIGYINPTTGYTKYNQKFKDKATLTA |
| | | | | DKSSSTAYMQLSSLTNVDSAVYYCTTGYSYFDYWGQGT |
| | | | | TVTVSSGGGGSGGGGSGGGGSDIELTQSPAIMSASPGE |
| | | | | KVTITCSASSSVNYMHWFQQKPGTSPKLWISSTSNLAS |
| | | | | GVPARFSGSGSGTSYSLTISRMEAEDAATYYCQQRSSY |
| | | | | PYTFGGGTKLQIKRAAAGAPVPYPDPLEPRAA |
| 17 | 101 | JB1-G&S-scFv2 | 329 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSQVKLQESGAELVRSGASVNLSCTASGFNIKDYYM |
| | | | | HWVKQRPEEGLEWIGYIDPESGETEYAPNFQGKATVTA |
| | | | | DTSSNTAYLHLSSLTSEDTTVYYCNAVIYYEYDGYALD |
| | | | | YWGQGTTVTVSSGGGGSGGGGSGGGGSDIELTQSPSSL |
| | | | | AVSAGEKVAMSCKSSQSLFNSRTRKNYLAWYQQKPGQS |
| | | | | PKVLIYWASTRESGVPDRFTGSGSGTDFTLTISSVQAE |
| | | | | DLAVYYCKQSYNLPTFGGGTKLEIK |
| 18 | 102 | JB1-G4S-scFv3 | 323 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSQVQLQQSGAELVRSGASVKLSCTASGFNIKDYYM |
| | | | | HWVKQRPEQGLEWIGWIDPENGDTEYAPKFQGKATMTA |
| | | | | DTSSNTAYLQLSSLASEDTAVYYCNFYGDALDYWGQGT |
| | | | | TVTVSSGGGGSGGGGSGGGGSDVLMTQTPLTLSVTIGQ |
| | | | | PASISCKSSQSLLDSDGKTYLNWLLQRPGQSPKRLIYL |
| | | | | VSKLDSGVPDRFTGSGSGTDFTLKINRVEAEDLGVYYC |
| | | | | WQGTHSPLTFGAGTKLEIK |
| 19 | 103 | JB1-G4S-scFv4 | 313 | MGKDYYQTLGLARGASDEEIKRAYRRQALRYHPDKNKE |
| | | | | PGAEEKFKEIAEAYDVLSDPRKREIFDRYGEEGLKGSG |
| | | | | GGGSQESGAELVRSGASVKLSCTTSGFNINDYYMHWVK |
| | | | | KRPEQGLEWIGRIDPENGDADMTRSSGVKATMTADTSS |
| | | | | NTAYLQLSSLTSEDTAVYYCNAGMDYWGQGTTVTVSSG |
| | | | | GGGSGGRASGGGGSDIELTQSPASLAVSLGQRATISCR |
| | | | | ASKSVSTSGYSYMHWNQQKPGQPPRLLIYLVSNLESGV |
| | | | | PARFSGSGSGTDFTLNIHPVEEEDAATYYCQHIRELTR |
| | | | | SEGGTKLEI |
| 20 | 104 | JB6-G4S-scFv1 | 330 | MVDYYEVLGVQRHASPEDIKKAYRKLALKWHPDKNPEN |
| | | | | KEEAERKEKQVAEAYEVLSDAKKRDIYDKYGGGGGSQV |
| | | | | KLQQSGAELAKPGASVKMSCKTSGYTFTSYWMNWVKQR |
| | | | | PGQGLEWIGYINPTTGYTKYNQKFKDKATLTADKSSST |
| | | | | AYMQLSSLTNVDSAVYYCTTGYSYFDYWGQGTTVTVSS |
| | | | | GGGGSGGGGSGGGGSDIELTQSPAIMSASPGEKVTITC |
| | | | | SASSSVNYMHWFQQKPGTSPKLWISSTSNLASGVPARF |
| | | | | SGSGSGTSYSLTISRMEAEDAATYYCQQRSSYPYTFGG |
| | | | | GTKLQIKRAAAGAPVPYPDPLEPRAA |
| 21 | 105 | JB6-G4S-scFv2 | 323 | MVDYYEVLGVQRHASPEDIKKAYRKLALKWHPDKNPEN |
| | | | | KEEAERKEKQVAEAYEVLSDAKKRDIYDKYGGGGGSQV |
| | | | | KLQESGAELVRSGASVNLSCTASGFNIKDYYMHWVKQR |
| | | | | PEEGLEWIGYIDPESGETEYAPNFQGKATVTADTSSNT |
| | | | | AYLHLSSLTSEDTTVYYCNAVIYYEYDGYALDYWGQGT |
| | | | | TVTVSSGGGGSGGGGSGGGGSDIELTQSPSSLAVSAGE |
| | | | | KVAMSCKSSQSLFNSRTRKNYLAWYQQKPGQSPKVLIY |
| | | | | WASTRESGVPDRFTGSGSGTDFTLTISSVQAEDLAVYY |
| | | | | CKQSYNLPTFGGGTKLEIK |
| 22 | 106 | JB6-GS-scFv3 | 317 | MVDYYEVLGVQRHASPEDIKKAYRKLALKWHPDKNPEN |
| | | | | KEEAERKFKQVAEAYEVLSDAKKRDIYDKYGGGGGSQV |
| | | | | QLQQSGAELVRSGASVKLSCTASGFNIKDYYMHWVKQR |
| | | | | PEQGLEWIGWIDPENGDTEYAPKFQGKATMTADTSSNT |
| | | | | AYLQLSSLASEDTAVYYCNFYGDALDYWGQGTTVTVSS |
| | | | | GGGGSGGGGSGGGGSDVLMTQTPLTLSVTIGQPASISC |
| | | | | KSSQSLLDSDGKTYLNWLLQRPGQSPKRLIYLVSKLDS |
| | | | | GVPDRFTGSGSGTDFTLKINRVEAEDLGVYYCWQGTHS |
| | | | | PLTFGAGTKLEIK |
| 23 | 107 | JB6-G4S-scFv4 | 307 | MVDYYEVLGVQRHASPEDIKKAYRKLALKWHPDKNPEN |
| | | | | KEEAERKFKQVAEAYEVLSDAKKRDIYDKYGGGGGSQE |
| | | | | SGAELVRSGASVKLSCTTSGENINDYYMHWVKKRPEQG |
| | | | | LEWIGRIDPENGDADMTRSSGVKATMTADTSSNTAYLQ |
| | | | | LSSLTSEDTAVYYCNAGMDYWGQGTTVTVSSGGGGSGG |
| | | | | RASGGGGSDIELTQSPASLAVSLGQRATISCRASKSVS |
| | | | | TSGYSYMHWNQQKPGQPPRLLIYLVSNLESGVPARFSG |
| | | | | SGSGTDFTLNIHPVEEEDAATYYCQHIRELTRSEGGTK |
| | | | | LEI |

### II. Nucleic Acids Encoding Fusion Protein Constructs

According to another aspect of the invention, provided are isolated nucleic acids comprising a polynucleotide sequence selected from (a) a polynucleotide encoding the fusion protein of any of the foregoing embodiments, or (b) the complement of the polynucleotide of (a). The present invention provides isolated nucleic acids encoding fusion proteins comprising the J domain and p53-binding domain, and sequences complementary to such nucleic acid molecules encoding the fusion proteins, including homologous variants thereof. In another aspect, the invention encompasses methods to produce nucleic acids encoding the fusion proteins disclosed herein, and sequences complementary to the nucleic acid molecules encoding fusion proteins, including homologous variants thereof. The nucleic acid according to this aspect of the invention can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In yet another aspect, disclosed is a method of producing a fusion protein comprising (a) synthesizing and/or assembling nucleotides encoding the fusion protein, (b) incorporating the encoding gene into an expression vector appropriate for a host cell, (c) transforming the appropriate host cell with the expression vector, and (d) culturing the host cell under conditions causing or permitting the fusion protein to be expressed in the transformed host cell, thereby producing the biologically-active fusion protein, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode the fusion proteins disclosed herein (or its complement) are used to generate recombinant DNA molecules that direct the expression of the fusion proteins in appropriate host cells. Several cloning strategies are suitable for performing the present invention, many of which is used to generate a construct that comprises a gene coding for a fusion protein of the present invention, or its complement. In some embodiments, the cloning strategy is used to create a gene that encodes a fusion protein of the invention, or their complement.

In certain embodiments, a nucleic acid encoding one or more fusion proteins is an RNA molecule, and can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In various embodiments, the nucleic acid is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from Rnases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly (A) sequence of between about 100 and about 1000 bases, between about 200 and about 500 bases, or between about 300 and about 400 bases. In one embodiment, the mRNA comprises a poly (A) sequence of about 65 bases, about 100 bases, about 200 bases, about 300 bases, about 400 bases, about 500 bases, about 600 bases, about 700 bases, about 800 bases, about 900 bases, or about 1000 or more bases. Poly(A) sequences can be modified chemically or enzymatically to modulate mRNA functionality such as localization, stability or efficiency of translation.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In certain embodiments, the nucleic acid sequence comprises a nucleotide sequence encoding the gene of interest (e.g., the fusion proteins comprising a J domain and a p53 binding domain) within a nucleic acid cassette. The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Illustrative ubiquitous expression control sequences suitable for use in particular embodiments include, but are not limited to, a cytomegalovirus (CMV) immediate early promoter, a viral simian virus 40 (SV40) (e.g early or late), a Moloney murine leukemia virus (MoMLV) LTR promoter, a Rous sarcoma virus (RSV) LTR, a herpes simplex virus (HSV) (thymidine kinase) promoter, H5, P7.5, and PI I promoters from vaccinia virus, an elongation factor 1-alpha (EFIa) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), ß-kinesin (b-KIN), the human ROSA 26 locus (Irions et al, Nature Biotechnology 25, 1477-1482 (2007)), a Ubiquitin C promoter (UBC), a phosphogly cerate kinase-1 (PGK) promoter, a cytomegalovirus enhancer/chicken ß-actin (CAG) promoter (Okabe et al. (1997) FEBS let. 407: 313-9), a b-actin promoter and a myeloproliferative sarcoma virus enhancer, negative control region deleted, dl587rev primer binding site substituted (MND) U3 promoter (Haas et al., Journal of Virology. 2003; 77(17): 9439-9450).

In one embodiment, at least one element may be used with the polynucleotides described herein to enhance the transgene target specificity and expression (See e.g., Powell et al. (2015) Discovery Medicine 19(102):49-57, the contents of which are herein incorporated by reference in its entirety) such as promoters. Promoters for which promote expression in most tissues include, but are not limited to, human elongation factor Ia-subunit (EFIa), immediate-early cytomegalovirus (CMV), chicken β-actin (CBA) and its derivative CAG, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, ηβ2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes include the myelin basic protein (MBP) promoter. Yu et al. (2011) Molecular Pain, 7:63, incorporated by reference in its entirety) evaluated the expression of eGFP under the CAG, EFIa, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015, incorporated by reference in its entirety) the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al, (2001) Gene Therapy, Vol. 8, 1539-1546; incorporated by reference in its entirety). Husain et al. (2009) Gene Therapy, incorporated by reference in its entirety) evaluated a HβH construct with a hGUSB promoter, a HSV-1LAT promoter and a NSE promoter and found that the H3H construct showed weaker expression than NSE in mice brain. Passini and Wolfe (J. Virol. 2001, 12382-12392, incorporated by reference in its entirety) evaluated the long term effects of the H3H vector following an intraventricular injection in neonatal mice and found that there was sustained expression for at least 1 year. Low expression in all brain regions was found by Xu et al. (2001) Gene Therapy, 8, 1323-1332; incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. 2007 and Raymond et al. 2004; incorporated by reference in its entirety).

### III. Vectors Comprising Nucleic Acids Encoding Fusion Proteins

Also provided is a vector comprising nucleic acid according to the invention. Such a vector preferably comprises additional nucleic acid sequences such as elements necessary for transcription/translation of the nucleic acid sequence encoding a phosphatase (for example promoter and/or terminator sequences). Said vectors can also comprise nucleic acid sequences coding for selection markers (for example an antibiotic) to select or maintain host cells transformed with said vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to an affected cell (e.g. neuronal cells) In one embodiment, the vector is an in vitro synthesized or synthetically prepared mRNA encoding a fusion protein comprising a J domain and a p53-binding domain. Illustrative examples of non-viral vectors include, but are not limited to mRNA, plasmids (e.g., DNA plasmids or RNA plasmids), transposons, cosmids, and bacterial artificial chromosomes.

Illustrative examples of vectors include, but are not limited to, a plasmid, autonomously replicating sequences, and transposable elements, e.g., piggyBac, Sleeping Beauty, Mosl, Tcl/mariner, Tol2, mini-Tol2, Tc3, MuA, Himar I, Frog Prince, and derivatives thereof. Additional Illustrative examples of vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Illustrative examples of viruses useful as vectors include, without limitation, retrovirus (including lentivirus), adenovirus, adeno-associated virus, herpesvirus (e.g., herpes simplex vims), poxvirus, baculovirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, and pLenti6.2/V 5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

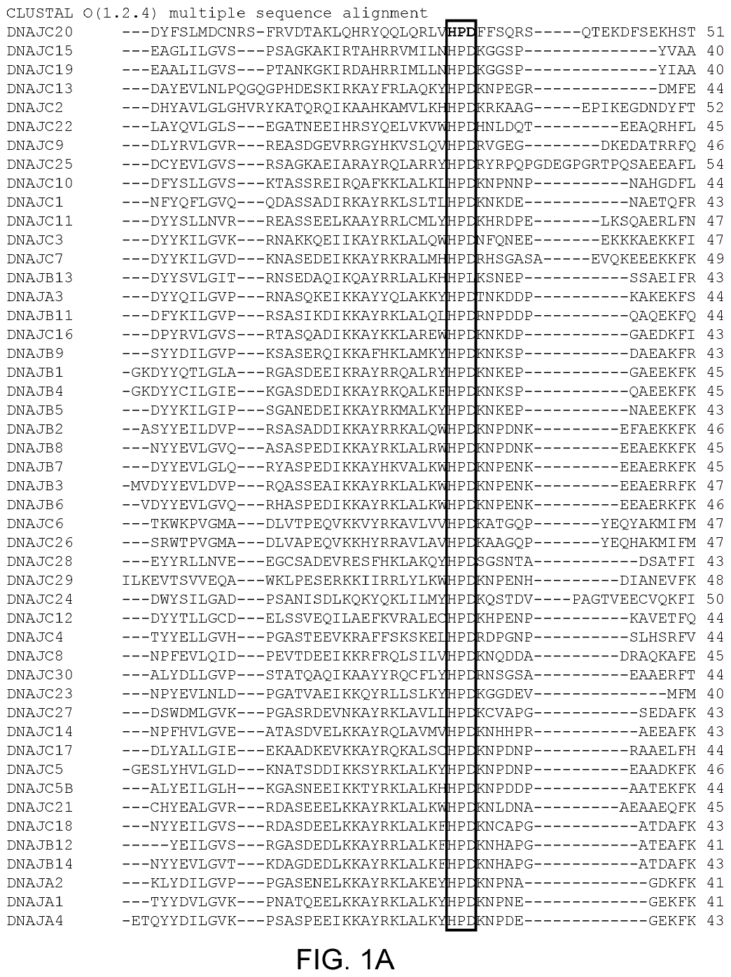

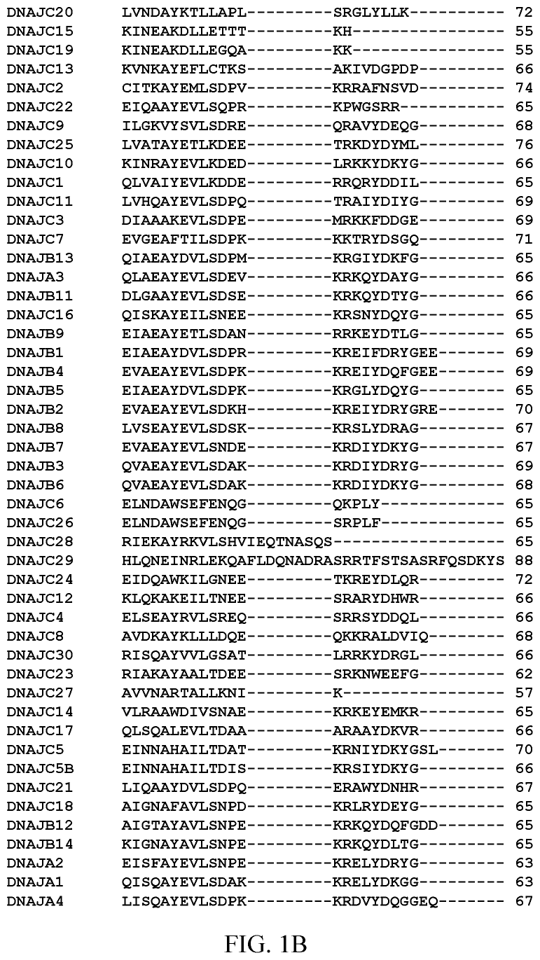

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Suitable recognition sites for the FLP recombinase include, but are not limited to: FRT (McLeod, et al., 1996), FI, F2, F3 (Schlake and Bode, 1994), FyFs (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme I Integrase, e.g., phi-c3I. The (pC3I SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by ϕ031 homodimers (Groth et al., 2000). The product sites, attL and attR, are effectively inert to further tpQA 1-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a fusion protein comprising a J domain and p53-binding domain. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding fusion protein comprising a J domain and a p53-binding domain are introduced into a cell, e.g., a neuronal cell, by transducing the cell with a recombinant adeno-associated virus (rAAV), comprising the one or more polynucleotides. AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2 (described, for example, in U.S. Pat. No. 6,962,815B2, which is incorporated herein by reference in its entirety), AAV3, AAV4, AAV5 (described, for example, in U.S. Pat. No. 7,479,554B2, which is incorporated herein by reference in its entirety), AAV6, AAV7, AAV8 (described, for example, in U.S. Pat. No. 7,282,199B2, which is incorporated herein by reference in its entirety), AAV9 (described, for example, in U.S. Pat. No. 9,737,618B2, which is incorporated herein by reference in its entirety), AAV rh10 (described, for example, in U.S. Pat. No. 9,790,472B2, which is incorporated herein by reference in its entirety) or AAV 10. In one embodiment, the vector of the present invention is encapsulated into a capsid selected from the group consisting of AAV2, AAV5, AAV8, AAV9 and AAV rh10. In one embodiment, the vector is encapsulated in AAV2. In one embodiment, the vector is encapsulated in AAV5. In one embodiment, the vector is encapsulated in AAV8. In one embodiment, the vector is encapsulated in AAV9. In still one embodiment, the vector is encapsulated in AAV rh10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

### IV. Delivery

In particular embodiments, one or more polynucleotides encoding a fusion protein comprising a J domain and p53-binding domain are introduced into a cell by non-viral or viral vectors. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, poly cation or lipid nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al., (2003) Gene Therapy. 10: 180-187; and Balazs et al., (20W) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion), by intrathecal injection, intracerebroventricular injection or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a fusion protein disclosed herein is administered directly to an organism for transduction of cells in vivo.

Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

In various embodiments, one or more polynucleotides encoding a fusion protein disclosed herein are introduced into a cell, for example, a neuronal cell or neuronal stem cell, by transducing the cell with a retrovirus, e.g., lentivirus, comprising the one or more polynucleotides. As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex vims (HSV) (thymidine kinase) promoters. In certain embodiments, lentiviral vectors are produced according to known methods. See e.g., Kutner et al., BMC Biotechnol. 2009; 9:10. Doi: 10.1186/1472-6750-9-10; Kutner et al., Nat. Protoc. 2009; 4(4):495-505. Doi: 10.1038/nprot.2009.22.

According to certain specific embodiments contemplated herein, most or all of the viral vector backbone sequences are derived from a lentivirus, e.g., HIV-I. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a fusion protein disclosed herein are introduced into a target cell by transducing the cell with an adenovirus comprising the one or more polynucleotides. Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad EIa, EIb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses EI proteins (Graham et al., 1977). Since the E3 region is dispensable from the adenovirus genome (Jones & Shenk, 1978), the current adenovirus vectors, with the help of 293 cells, carry foreign DNA in either the EI, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7: 1083-9 (1998)).

In various embodiments, one or more polynucleotides encoding a fusion protein of the invention are introduced into the target cell of a subject by transducing the cell with a herpes simplex virus, e.g., HSV-I, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which is incorporated by reference herein in its entirety.

### V. Cells Expressing the Fusion Protein

In yet another aspect, the invention provides for cells expressing the fusion proteins described herein. Cells can be transfected with a vector encoding the fusion protein as described herein above. In one embodiment, the cell is a prokaryotic cell. In another embodiment, the cell is a eukaryotic cell. In still another embodiment, the cell is a mammalian cell. In a particular embodiment, the cell is a human cell. In another embodiment, the cell is a human cell that is derived from a patient that suffers from, or is at risk of suffering from, a p53-mediated neoplasia including, but not limited to, cancers and other neoplasms, including but not limited to: malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome. The cell can be a hepatocyte or a muscle cell.

Cells expressing the fusion protein can be useful in producing the fusion protein. In this embodiment, the cells are transfected with a vector overexpressing the fusion protein. The fusion protein may optionally contain an epitope, for example, a human Fc domain or a FLAG epitope, as described herein above, that would facilitate the purification (using a Protein A- or anti-FLAG antibody column, respectively). The epitope may be connected to the rest of the fusion protein via a linker or a protease substrate sequence such that, during or after purification, the epitope can be removed from the fusion protein.

In an alternative embodiment, cells expressing a secreted form of the fusion protein can be used. For example, fusion protein constructs can be designed having a signal sequence on the N-terminal end. Representative signal sequences are shown below in Table 7.

TABLE 7
Representative Signal Sequences

| SEQ ID NO: | SEQUENCE |
|---|---|
| 94 | MGVKVLFALICIAVAEA |
| 95 | MAPVQLLGLLVLELPAMRC |
| 96 | MAVLGLLFCLVTFPSCVLS |

Therefore, in one embodiment, the fusion protein comprises a signal sequence and a fusion protein, wherein the signal sequence is selected from the group consisting of SEQ ID NOs: 94-96, and a fusion protein comprising a J domain and a p53 binding domain. In one embodiment, the signal sequence is selected from the group consisting of SEQ ID NOs: 94-96, and a fusion protein is selected from the group consisting of SEQ ID NOs: 80-91, 100-107. In another embodiment, the fusion protein comprises the signal sequence of SEQ ID NO: 94, and the fusion protein selected from the group consisting of SEQ ID NOs: 80-91, 100-107. In another embodiment, the fusion protein comprises the signal sequence of SEQ ID NO: 95, and the fusion protein selected from the group consisting of SEQ ID NOs: 80-91, 100-107. In another embodiment, the fusion protein comprises the signal sequence of SEQ ID NO: 96, and the fusion protein selected from the group consisting of SEQ ID NOs: 80-91, 100-107. Cells expressing the fusion protein constructs with the signal sequence can be administered to a subject, for example a human subject (e.g., a patient having or at risk of suffering from a p53 disorder). The fusion protein is secreted from the cells, which help restore p53 functions and/or reduce p53 aggregation.

As described herein above, in certain embodiments, the fusion protein can further comprise a cell-penetrating peptide. A cell expressing a fusion protein comprising a signal sequence and a cell-penetrating peptide would be capable of secreting the fusion protein, devoid of the signal sequence. The secreted fusion protein, also comprising the cell-penetrating peptide, would then be capable of entering nearby cells, and have the potential to restore p53 functions and/or reduce p53 aggregation in those cells.

### VI. Methods of Use

In another aspect, the invention provides a method for achieving a beneficial effect in disorders and/or in a p53 disorder, disorder or condition mediated by mutant p53. The p53 disorder is a cancer. The cancer can include any of: malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

In some embodiments, the invention provides methods for treating a subject, such as a human, with a p53 disease, disorder or condition comprising the step of administering to the subject a therapeutically- or prophylactically-effective amount of a fusion protein, a nucleic acid encoding such fusion protein, or a viral vector encoding such fusion protein described herein, wherein said administration results in the improvement of one or more biochemical or physiological parameters or clinical endpoints associated with the p53 disease, disorder or condition.

In other embodiments, the invention provides for a method of restoration of p53 function and/or reduce p53 aggregation in a cell. The cell can be a cultured cell or an isolated cell. The cell can also be from a subject, for example, a human subject. In one embodiment, the human subject is suffering from, or is at risk of suffering from a p53 disorder disease, including one or more types of cancers.

Activity of p53 proteins can be detected in a number of ways. In one example, transctiptional activity of p53 can be monitored by downstream genes such as p21. In another embodiment, p53 function is monitored by reporter gene assay comprising artificial reporter constructs.

Therefore, in one embodiment, the method comprises contacting the cell with an amount of the fusion protein or a nucleic acid, vector, or viral particle encoding the fusion protein effective to restore p53 activity by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

As shown below in Example 1, expression of fusion proteins comprising a J domain and a p53 binding domain has been found to restore the level of p21 expression. As such, in another embodiment, the method comprises contacting the cell with an amount of the fusion protein, a cell expressing the fusion protein, a nucleic acid, vector, or viral particle encoding the fusion protein effective to restore the function of p53 proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

### VII. Pharmaceutical Compositions

The compositions contemplated herein may comprise one or more fusion protein comprising a J domain and p53-binding domain, polynucleotides encoding such fusion proteins, vectors comprising same, genetically modified cells, etc., as contemplated herein. Compositions include, but are not limited to pharmaceutical compositions. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier”, “diluent” or “excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

### VIII. Dosages

The dosage of the compositions (e.g., a composition including a fusion protein construct, nucleic acid or gene therapy viral particle) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition is a prophylactically or a therapeutically effective amount.

### IX. Kits

Kits including (a) a pharmaceutical composition including a fusion protein construct, nucleic acid encoding such fusion protein, or viral particle encompassing such nucleic acid that restore p53 functions in a cell or subject described herein, and (b) a package insert with instructions to perform any of the methods described herein are contemplated. In some aspects, the kit includes (a) a pharmaceutical composition including a composition described herein that restoers p53 functions in a cell or subject described herein, (b) an additional therapeutic agent, and (c) a package insert with instructions to perform any of the methods described herein.

### EXAMPLES

To test whether J domains can be specifically engineered to facilitate the proper folding of mutant proteins, we designed and tested a number of fusion protein constructs designed to target p53 proteins.

### Example 1: Fusion Protein Design

### A. Methods

### General Techniques and Materials

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al., eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N J, 2000, the contents of which are incorporated in their entirety herein by reference. HEK-293 cells (human embryonic kidney cells) were purchased from the American Type Culture Collection (Manassas, VA). Anti-FLAG antibody was purchased from Thermo Fisher Scientific. Rabbit anti-GFP antibody was purchased from GenScripts (Piscataway, NJ). For ease of purification and characterization, some of the fusion protein constructs used in this Example 1 contain, in addition to the sequences provided in SEQ ID NOs: 80-91, the FLAG epitope of SEQ ID NO:69 at either the C-terminus or N-terminus of the protein, in addition to a short linker sequence.

### Expression and Detection of Proteins in HCT-116 Cells

Expression vector plasmids encoding various protein constructs were transfected into HCT-116 cells with Lipofectamine 3000 transfection reagent (Thermo Fisher Scientific). Cell lysates were analyzed for expressed proteins using immunoblot assays. Samples of culture media were centrifuged to remove debris prior to analysis. Cells were lysed in a lysis buffer (10 mM Tris-HCl, pH 8.0, 150 mM NaCl, 10 mM EDTA, 2% SDS) containing 2 mM PMSF and protease cocktail (Complete Protease Inhibitor Cocktail; Sigma). After brief sonication, the samples were analyzed for expressed proteins using immunoblot assays. For immunoblot analysis, samples were boiled in an SDS-sample buffer and run on polyacrylamide electrophoresis. Thereafter, the separated protein bands were transferred to a PVDF membrane.

Expressed proteins were detected using a chemiluminescent signal. Briefly, blots were reacted with a primary antibody capable of binding the particular protein (e.g., p21). After rinsing away the unreacted primary antibody, a secondary, enzyme-linked antibody (e.g., HRP-linked anti-IgG antibody) was allowed to react with the primary antibody molecules bound to the blots. Following rinsing, a chemiluminescent reagent was added, and the resultant chemiluminescent signals in the blots were captured on X-ray film.

### ELISA

HCT-116 cells were homogenized in lysis buffer (50 mM Tris, pH8.0, 120 mM NaCl, 0.5% NP-40) supplemented with protease inhibitor cocktail, 2 mM PMSF, 10 mM NaF, and 2 mM Na3VO4. After brief sonication, debris were removed by centrifugation. Protein concentration is measured by BCA assay kit (Pierce). The supernatant was applied to ELISA (Human Total p21/CIP1/CDKN1A DuoSet IC ELISA: R&D Systems) with manufacture's standard protocol.

### Cytotoxicity Assay (LDH Assay)

HCT-116 cells or HCT-116 cells stably expressing p53R249S were seeded onto 48-well culture plate. At Day 1, culture medium was replaced with fresh medium containing 8 μg/ml polybrene and lentivirus particles to express Construct-2 or control lentivirus encoding no substantial gene. After 72 hours, culture medium was replaced with fresh medium containing 1 uM Doxorubicin and cells were cultured for another 12 hours. For some study, Construct-2 was expressed by lipofection. 24 hours after transfection, culture medium was replaced with fresh medium and cells were cultured for another 24-48 hours. Culture medium was then harvested and applied to LDH assay (LDH-Cytox™ Assay Kit: BioLegend: 426401) with manufactures' protocol.

### B. Reporter Constructs

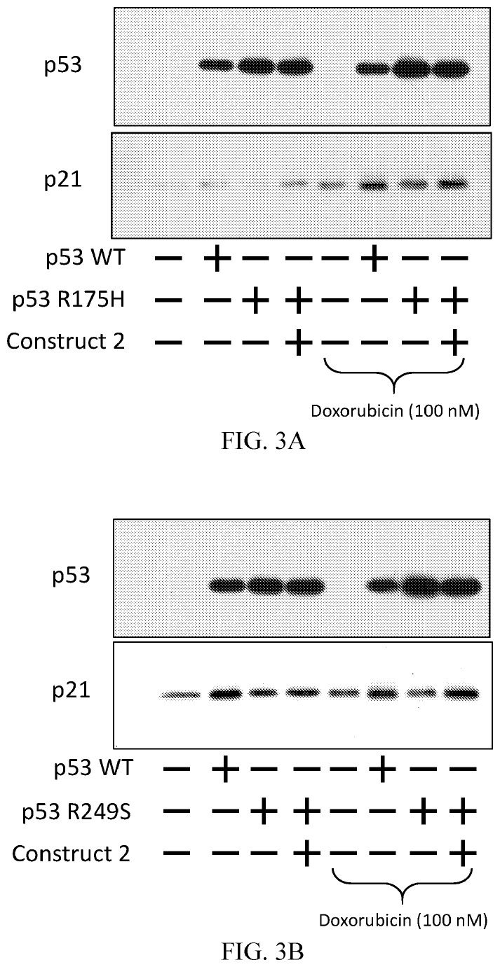

We first investigated whether the fusion molecule of the present invention targeting p53 restores its function in cultured cells. To this end, we generated p53 constructs, including the wild-type p53, as well as p53 containing R175H and R249S mutations which is known as “conformational mutant” (See Table 8 below). HCT-116 cells were cultured and transfected with the plasmids encoding the wild-type p53 or the p53 mutant, containing R175H (SEQ ID NO: 98) or R249S (SEQ ID NO:99). As shown in FIG. 3, we found that the downstream induction of p21 by p53 was abolished when mutant p53 containing the R175H (left) or R249S (right) was expressed instead of wild-type p53 (compare, for example, lanes 2 vs 3).

TABLE 8
p53 Reporter Constructs

| Construct | | | |
|---|---|---|---|
| Name | SEQ ID NO: | Length | Sequence |
| p53WT | 97 | 393 | MEEPQSDPSVEPPLSQETFSDLWKLLPENNVLSPLPSQAMDDL |
| | | | MLSPDDIEQWFTEDPGPDEAPRMPEAAPPVAPAPAAPTPAAP |
| | | | APAPSWPLSSSVPSQKTYQGSYGFRLGFLHSGTAKSVTCTYSPAL |
| | | | NKMFCQLAKTCPVQLWVDSTPPPGTRVRAMAIYKQSQHMTEV |
| | | | VRRCPHHERCSDSDGLAPPQHLIRVEGNLRVEYLDDRNTFRHSV |
| | | | VVPYEPPEVGSDCTTIHYNYMCNSSCMGGMNRRPILTIITLEDSS |
| | | | GNLLGRNSFEVRVCACPGRDRRTEEENLRKKGEPHHELPPGSTK |
| | | | RALPNNTSSSPQPKKKPLDGEYFTLQIRGRERFEMFRELNEALEL |
| | | | KDAQAGKEPGGSRAHSSHLKSKKGQSTSRHKKLMFKTEGPDSD |
| p53R175H | 98 | 393 | MEEPQSDPSVEPPLSQETFSDLWKLLPENNVLSPLPSQAMDDL |
| | | | MLSPDDIEQWFTEDPGPDEAPRMPEAAPPVAPAPAAPTPAAP |
| | | | APAPSWPLSSSVPSQKTYQGSYGFRLGFLHSGTAKSVTCTYSPAL |
| | | | NKMFCQLAKTCPVQLWVDSTPPPGTRVRAMAIYKQSQHMTEV |
| | | | VRHCPHHERCSDSDGLAPPQHLIRVEGNLRVEYLDDRNTFRHSV |
| | | | VVPYEPPEVGSDCTTIHYNYMCNSSCMGGMNRRPILTIITLEDSS |
| | | | GNLLGRNSFEVRVCACPGRDRRTEEENLRKKGEPHHELPPGSTK |
| | | | RALPNNTSSSPQPKKKPLDGEYFTLQIRGRERFEMFRELNEALEL |
| | | | KDAQAGKEPGGSRAHSSHLKSKKGQSTSRHKKLMFKTEGPDSD |
| p53R249S | 99 | 393 | MEEPQSDPSVEPPLSQETFSDLWKLLPENNVLSPLPSQAMDDL |
| | | | MLSPDDIEQWFTEDPGPDEAPRMPEAAPPVAPAPAAPTPAAP |
| | | | APAPSWPLSSSVPSQKTYQGSYGFRLGFLHSGTAKSVTCTYSPAL |
| | | | NKMFCQLAKTCPVQLWVDSTPPPGTRVRAMAIYKQSQHMTEV |
| | | | VRRCPHHERCSDSDGLAPPQHLIRVEGNLRVEYLDDRNTFRHSV |
| | | | VVPYEPPEVGSDCTTIHYNYMCNSSCMGGMNRSPILTIITLEDSS |
| | | | GNLLGRNSFEVRVCACPGRDRRTEEENLRKKGEPHHELPPGSTK |
| | | | RALPNNTSSSPQPKKKPLDGEYFTLQIRGRERFEMFRELNEALEL |
| | | | KDAQAGKEPGGSRAHSSHLKSKKGQSTSRHKKLMFKTEGPDSD |

### C. Fusion Protein Constructs

To determine whether the fusion proteins of the present invention could be used to restore p53 function, an initial experiment was first conducted by co-expression of a fusion protein comprising a J-domain sequence derived from a human Hsp40 J-domain protein, conjugated to the peptide sequence that recognizes mutant p53 (data not shown). Plasmid encoding Construct 2 (JB1-CDB3) was transfected with reporter construct (p53R175H or p53R249S) into HCT-116 cells. After the expression, cells were lysed in lysis buffer, followed by immnoblotting assay using anti-p21 antibody or anti-p53 antibody. Expression of wild-type p53 resulted in induction of p21 while neither mutant (R175H or R249S) failed to do so. Surprisingly, cells expressing Construct-2 showed substantial restoration of p21 expression but p53 protein level itself has not changed significantly (FIG. 3).

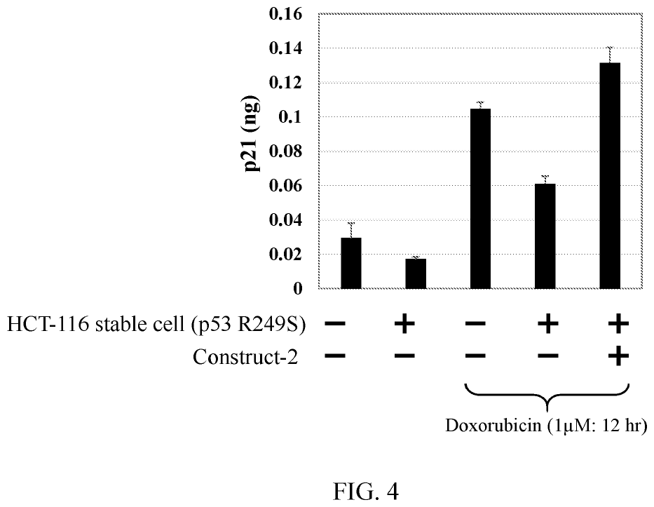

To verify the results further, stable cells expressing mutant p53 (R249S) were established by standard method. Original HCT-116 cells or HCT-116 cells stably expressing p53R249S were cultured and incubated with lentivirus to express Construct-2. After the incubation, cells were stimulated by 1 uM doxorubicin for 12 hours. Cells were lysed and protein concentration was measured by BCA assay. Equal amount of cell lysate was applied to ELISA to quantify the p21 expression. As shown in FIG. 4, cells stably expressing mutant p53 kept the lower p21 expression levels (first bar vs second bar). Incubation with doxorubicin strongly induce the expression of p21 (third bar) but less induction in cells expressing mutant p53 (fourth bar). Surprisingly, Construct-2 completely resorted the induction of p21 (fifth bar).

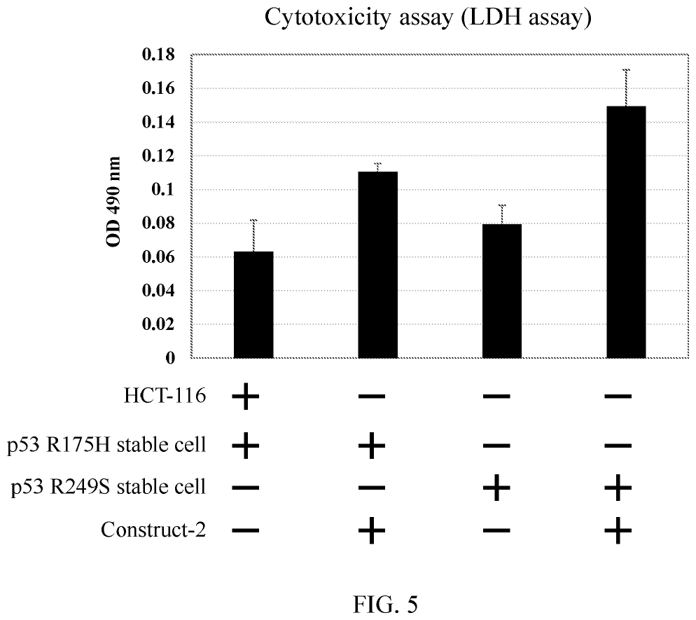

Finally, cytotoxic activity of p53 was assessed. HCT-116 cells stably expressing either R175H or R249S p53 mutant were cultured and Construct-2 was expressed using lentivirus as described above. After expression, cells were cultured in the presence of 1 μM doxorubicin for another 12 hours. Culture medium was harvested and LDH cytotoxicity assay was performed. As shown in FIG. 5, cells expressing Construct-2 were more susceptible to the cytotoxic effects of doxorubicin. These data suggested that p53 conformational problem could be resolved using the HSP70-mediated pathway.

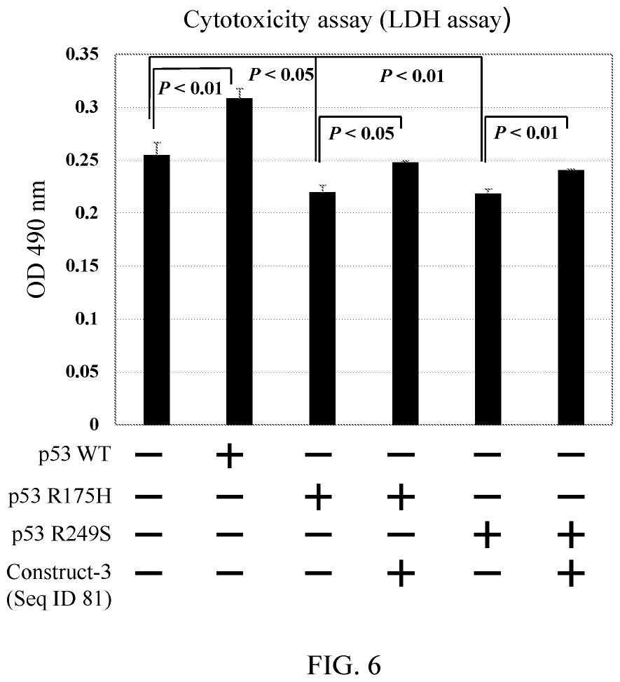

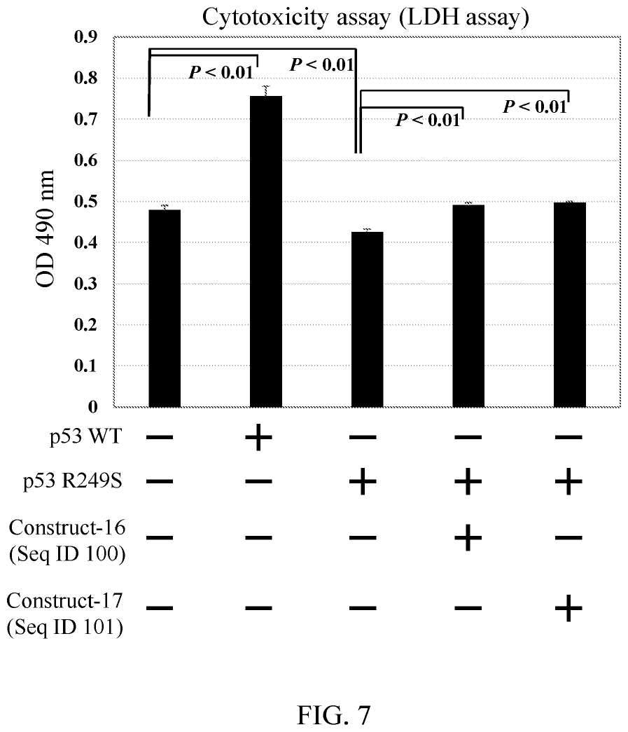

Similarly, additional constructs were generated and tested. Cells expressing either the R175H or R249S mutant forms of p53 showed the less cytotoxic effects compared to wild-type p53 while either Construct 3 (FIG. 6), Construct 16 or Construct 17 (FIG. 7) partially restored the cytotoxic effects.

We then designed a series of fusion protein constructs, as depicted in Table 9.

TABLE 9
Fusion Protein Constructs and Controls

| Construct | SEQ | | |
|---|---|---|---|
| No | ID NO: | Construct Name | Notes |
| 1 | 79 | J-domain only | Control, containing the J domain from |
| | | | human DnaJB1 |
| 2 | 80 | JB1-CDB3 | DnaJB1 J domain fused to CDB3, which |
| | | | binds p53 |
| 3 | 81 | JB1-p53BP | DnaJB1 J domain fused to p53BP, which |
| | | | binds p53 |
| 4 | 82 | JB1-scFv1 (ME1) | DnaJB1 J domain fused to scFv1, which |
| | | | binds p53 |
| 5 | 83 | JB1-scFv2 (scFv11D3) | DnaJB1 J domain fused to scFv2, which |
| | | | binds p53 |
| 6 | 84 | JB1-scFv3 (scFv421) | DnaJB1 J domain fused to scFv3, which |
| | | | binds p53 |
| 7 | 85 | JB1-scFv4 (Pab421) | DnaJB1 J domain fused to scFv4, which |
| | | | binds p53 |
| 8 | 86 | JB6-CDB3 | Same as construct 2, except using the |
| | | | human DnaJB6 J domain. |
| 9 | 87 | JB6-p53BP | Same as construct 3, except using the |
| | | | human DnaJB6 J domain |
| 10 | 88 | JB6-scFv1 | Same as construct 4, except using the |
| | | | human DnaJB6 J domain |
| 11 | 89 | JB6-scFv2 | Same as construct 5, except using the |
| | | | human DnaJB6 J domain |
| 12 | 90 | JB6-scFv3 | Same as construct 6, except using the |
| | | | human DnaJB6 J domain |
| 13 | 91 | JB6-scFv4 | Same as construct 7, except using the |
| | | | human DnaJB6 J domain |
| 16 | 100 | JB1-G4S-scFv1 (ME1) | Same as construct 4, except for the |
| | | | additional GGGGS linker between the J |
| | | | domain and p53-binding domain |
| 17 | 101 | JB1-G4S-scFv2 (scFv11D3) | Same as construct 5, except for the |
| | | | additional GGGGS linker between the J |
| | | | domain and p53-binding domain |
| 18 | 102 | JB1-G4S-scFv3 (scFv421) | Same as construct 6, except for the |
| | | | additional GGGGS linker between the J |
| | | | domain and p53-binding domain |
| 19 | 103 | JB1-G4S-scFv4 (Pab421) | Same as construct 7, except for the |
| | | | additional GGGGS linker between the J |
| | | | domain and p53-binding domain |
| 20 | 104 | JB6-G4S-scFv1 (ME1) | Same as construct 10, except for the |
| | | | additional GGGGS linker between the J |
| | | | domain and p53-binding domain |
| 21 | 105 | JB6-G4S-scFv2 (scFv11D3) | Same as construct 11, except for the |
| | | | additional GGGGS linker between the J |
| | | | domain and p53-binding domain |
| 22 | 106 | JB6-G4S-scFv3 (scFv421) | Same as construct 12, except for the |
| | | | additional GGGGS linker between the J |
| | | | domain and p53-binding domain |
| 23 | 107 | JB6-G4S-scFv4 (Pab421) | Same as construct 13, except for the |
| | | | additional GGGGS linker between the J |
| | | | domain and p53-binding domain |

### Example 2: AAV Vectors Encoding Fusion Protein Constructs

An exemplary gene therapy vector is constructed by an AAV9 vector bearing a codon-optimized cDNA encoding the fusion protein constructs of Table 9. The cDNA encoding the construct is located downstream of the Kozak sequence and is polyadenylated by the bovine growth hormone polyadenylation (BGHpA) signal. The entire cassette is flanked by two non-coding terminal inverted sequences of AAV-2.

Recombinant AAV vector is prepared using a baculovirus expression system similar to that described above (Urabe et al., 2002, Unzu et al., 2011 (reviewed in Kotin, 2011)). Briefly, three recombinant baculoviruses, one encoding REP for replication and packaging, one encoding CAP-5 for the capsid of AAV9, and one having an expression cassette is used to infect SF9 insect cells. Purification is performed using AVB Sepharose high speed affinity media (GE Healthcare Life Sciences, Piscataway, NJ). Vectors are titrated using QPCR with the primer-probe combination for the transgene and titers are expressed as genomic copies per ml (GC/ml). The titer of the vector is approximately between 8×10¹³ to 2×10¹⁴ GC/ml.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. Therefore, in a first aspect, disclosed herein is an isolated fusion protein comprising a J domain of a J protein and a p53-binding domain.

2. The fusion protein of claim 1, wherein the J domain of a J protein is of eukaryotic origin.

3. The fusion protein of any one of claim 1-claim 2, wherein the J domain of a J protein is of human origin.

4. The fusion protein of any one of claim 1-claim 3, wherein the J domain of a J protein is cytosolically localized.

5. The fusion protein of any one of claim 1-claim 4, wherein the J domain of a J protein is selected from the group consisting of SEQ ID Nos: 1-50.

6. The fusion protein of any one of claim 1-claim 5, wherein the J domain comprises the sequence selected from the group consisting of SEQ ID NOs: 1, 5, 6, 10, 16, 24, 25, 31 and 49.

7. The fusion protein of any one of claim 1-claim 6, wherein the J domain comprises the sequence of SEQ ID NO: 5.

8. The fusion protein of any one of claim 1-claim 6, wherein the J domain comprises the sequence of SEQ ID NO: 10.

9. The fusion protein of any one of claim 1-claim 6, wherein the J domain comprises the sequence of SEQ ID NO: 16.

10. The fusion protein of any one of claim 1-claim 6, wherein the J domain comprises the sequence of SEQ ID NO: 25.

11. The fusion protein of any one of claim 1-claim 6, wherein the J domain comprises the sequence of SEQ ID NO: 31.

12. The fusion protein of any one of claim 1-claim 11, wherein the p53-binding domain has a K_{D} for p53 of 1 μM or less, for example, 300 nM or less, 100 nM or less, 30 nM or less, 10 nM or less, for example when measured using an ELISA assay.

13. The fusion protein of any one of claim 1-claim 12, wherein the p53-binding domain comprises the sequence selected from the group consisting of SEQ ID NOs: 51-56.

14. The fusion protein of any one of claim 1-claim 13, wherein the p53-binding domain comprises the sequence of SEQ ID NO:51-53.

15. The fusion protein of any one of claim 1-claim 13, wherein the p53-binding domain comprises the sequence of SEQ ID NO:51.

16. The fusion protein of any one of claim 1-claim 13, wherein the p53-binding domain comprises the sequence of SEQ ID NO:52.

17. The fusion protein of any one of claim 1-claim 16, comprising a plurality of p53-binding domains.

18. The fusion protein of any one of claim 1-claim 17, consisting of two p53-binding domains.

19. The fusion protein of any one of claim 1-claim 18, consisting of three p53-binding domains.

20. The fusion protein of any one of claim 1-claim 19, comprising one of the following constructs:

| | |
|---|---|
| a. | DNAJ-X-T, |
| b. | DNAJ-X-T-X-T, |
| c. | DNAJ-X-T-X-T-X-T, |
| d. | T-X-DNAJ, |
| e. | T-X-T-X-DNAJ, |
| f. | T-X-T-X-T-X-DNAJ, |
| g. | T-X-DNAJ-X-T, |
| h. | T-X-DNAJ-X-T-X-T, |
| i. | T-X-T-X-DNAJ-X-TT, |
| j. | T-X-T-X-DNAJ-X-T-X-T-X-T, |
| k. | T-X-T-X-T-X-DNAJ-X-T, |
| l. | T-X-T-X-T-X-DNAJ-X-T-X-T, |
| m. | T-X-T-X-T-X-DNAJ-X-T-X-T-X-T, |
| n. | DnaJ-X-DnaJ-X-T-X-T, |
| o. | T-X-DnaJ-X-DnaJ, |
| p. | T-X-T-X-DnaJ-X-DnaJ, and |

q. wherein,
r. T is a p53-binding domain,
s. DNAJ is a J domain of a J protein, and
t. X is an optional linker.

21. The fusion protein of any one of claim 1-claim 20, wherein the fusion protein comprises the J domain sequence of SEQ ID NO: 5 and the p53-binding domain sequence of SEQ ID NO: 52.

22. The fusion protein of any one of claim 1-claim 21, wherein the fusion protein comprises the J domain sequence of SEQ ID NO: 5 and two copies of the p53-binding domain sequence of SEQ ID NO: 52.

23. The fusion protein of any one of claim 1-claim 22, wherein the fusion protein comprises the sequence selected from the group consisting of SEQ ID NOs: 80-91, 100-107.

24. The fusion protein of any one of claim 1-claim 23, wherein the fusion protein comprises the sequence selected from the group consisting of SEQ ID NOs: 80.

25. The fusion protein of any one of claim 1-claim 23, wherein the fusion protein comprises the sequence of SEQ ID NO: 81.

26. The fusion protein of any one of claim 1-claim 23, wherein the fusion protein comprises the sequence of SEQ ID NO: 82.

27. The fusion protein of any one of claim 1-claim 23, wherein the fusion protein comprises the sequence of SEQ ID NO: 83.

28. The fusion protein of any one of claim 1-claim 23, wherein the fusion protein comprises the sequence of SEQ ID NO: 84.

29. The fusion protein of any one of claim 1-claim 23, wherein the fusion protein comprises the sequence of SEQ ID NO: 85.

30. The fusion protein of any one of claim 1-claim 23, wherein the fusion protein comprises the sequence of SEQ ID NO: 86.

31. The fusion protein of any one of claim 1-claim 23, wherein the fusion protein comprises the sequence of SEQ ID NO: 87.

32. The fusion protein of any one of claim 1-claim 23, wherein the fusion protein comprises the sequence of SEQ ID NO: 88.

33. The fusion protein of any one of claim 1-claim 23, wherein the fusion protein comprises the sequence of SEQ ID NO: 89.

34. The fusion protein of any one of claim 1-claim 23, wherein the fusion protein comprises the sequence of SEQ ID NO: 90.

35. The fusion protein of any one of claim 1-claim 23, wherein the fusion protein comprises the sequence of SEQ ID NO: 91.

36. The fusion protein of any one of claim 1-claim 35, further comprising a targeting reagent.

37. The fusion protein of any one of claim 1-claim 36, further comprising an epitope.

38. The fusion protein of E37, wherein the epitope is a polypeptide selected from the group consisting of SEQ ID NOs:68-74.

39. The fusion protein of any one of claim 1-claim 38, further comprising a cell-penetrating agent.

40. The fusion protein of claim 39, wherein the cell-penetrating agent is selected from the group consisting of SEQ ID NOs: 75-78.

41. The fusion protein of any one of claim 1-claim 40, further comprising a signal sequence.

42. The fusion protein of claim 41, wherein the signal sequence comprises the peptide sequence selected from the group consisting of SEQ ID NOs: 94-96.

43. The fusion protein of any one of claim 1-claim 42, which is capable of restoring p53 functions in a cell.

44. The fusion protein of any one of claim 1-claim 43, which is capable of reducing p53-mediated neoplasm.

45. A nucleic acid sequence encoding the fusion protein of any one of claim 1-claim 44.

46. The nucleic acid sequence of claim 45, wherein said nucleic acid is DNA.

47. The nucleic acid sequence of any one of claim 45, wherein said nucleic acid is RNA.

48. The nucleic acid sequence of any one of claim 40-claim 42, wherein said nucleic acid comprises at least one modified nucleic acid.

49. The nucleic acid sequence of any one of claim 45-claim 48, further comprising a promoter region, 5′ UTR, 3′ UTR such as poly(A) signal.

50. The nucleic acid sequence of claim 49, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.

51. A vector comprising the nucleic acid sequence of any one of claim 45-claim 50.

52. The vector of claim 51, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

53. The vector of claim 51 or claim 52, wherein the vector is an AAV.

54. A virus particle comprising a capsid and the vector of any one of claim 51-claim 53.

55. The virus particle of claim 54, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

56. The virus particle of claim 54 or claim 55, wherein the capsid is selected from the group consisting of AAV2, AAV5, AAV8, AAV9 and AAVrh10.

57. The virus particle of any one of claim 54-claim 56, wherein the capsid is AAV2.

58. The virus particle of any one of claim 54-claim 56, wherein the capsid is AAV5.

59. The virus particle of any one of claim 54-claim 56, wherein the capsid is AAV8.

60. The virus particle of any one of claim 54-claim 56, wherein the capsid is AAV9.

61. The virus particle of any one of claim 54-claim 56, wherein the capsid is AAV rh10.

62. A pharmaceutical composition comprising an agent selected from the group consisting of the fusion protein of any one of claim 1-claim 39, a cell expressing the fusion protein of claim 1-claim 39, the nucleic acid of any one of claim 40-claim 45, the vector of any one of claim 46-claim 48, the virus particle of any one of claim 49-claim 56, and a pharmaceutically acceptable carrier or excipient.

63. A method of reducing toxicity of a p53 protein in a cell, comprising contacting said cell with an effective amount of one or more agents selected from the group consisting of the fusion protein of any one of claim 1-claim 34, a cell expressing the fusion protein of claim 1-claim 34, the nucleic acid of any one of claim 45-claim 50, the vector of any one of claim 51-claim 53, the virus particle of any one of claim 54-claim 61, and the pharmaceutically composition of claim 62.

64. The method of claim 63, wherein the cell is in a subject.

65. The method of claim 64, wherein the subject is a human.

66. The method of any one of claim 64-claim 65, wherein subject is identified as having cancer.

67. The method of claim 66, wherein the cancer is selected from the group consisting malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

68. The method of any one of claim 63-claim 67, wherein there is a reduction in the amount of aberrant p53 protein in the cell when compared with a control cell.

69. A method of treating, preventing, or delaying the progression of a cancer in a subject in need thereof, the method comprising administering an effective amount of one or more agents selected from the group consisting of the fusion protein of any one of claim 1-claim 34, a cell expressing the fusion protein of claim 1-claim 34, the nucleic acid of any one of claim 45-claim 50, the vector of any one of claim 51-claim 53, the virus particle of any one of claim 54-claim 61, and the pharmaceutically composition of claim 62.

70. The method of claim 69, wherein the p53 disease is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

71. Use of one or more of the fusion protein of any one of claim 1-claim 34, a cell expressing the fusion protein of claim 1-claim 34, the nucleic acid of any one of claim 45-claim 50, the vector of any one of claim 51-claim 53, the virus particle of any one of claim 54-claim 61, and the pharmaceutically composition of claim 62, in the preparation of a medicament useful for the prevention or delay of progression of a p53 disease in a subject.

72. The use of claim 71, wherein the p53 disease is cancer.

73. The Use of claim 72, wherein the cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.
